(19) United States
(12) Patent Application Publication
(10) Pub. No.: US 2016/0024591 A1
(43) Pub. Date: Jan. 28, 2016

(54) METHODS AND COMPOSITIONS FOR CORRELATING GENETIC MARKERS WITH RISK OF AGGRESSIVE PROSTATE CANCER

(71) Applicants: WAKE FOREST UNIVERSITY HEALTH SCIENCES, Winston-Salem, NC (US); THE JOHNS HOPKINS UNIVERSITY, Baltimore, MD (US)

(72) Inventors: Jianfeng Xu, Clemmons, NC (US); Wennuan Liu, Clemmons, NC (US); William B. Isaacs, Glyndon, MD (US)

(21) Appl. No.: 14/775,969

(22) PCT Filed: Mar. 14, 2014
(86) PCT No.: PCT/US2014/028371

§ 371 Date: Sep. 14, 2015

Related U.S. Application Data
(60) Provisional application No. 61/785,636, filed on Mar. 14, 2013.

(51) Int. Cl. C12Q 1/68; G06F 19/00

(52) U.S. Cl. CPC C12Q 1/6886; G06F 19/3456; C12Q 2600/156

(57) ABSTRACT

The present invention provides a method of identifying a subject as having an increased risk of having or developing aggressive prostate cancer, comprising detecting in the subject the presence of various genetic markers associated with an increased risk of having or developing aggressive prostate cancer.

### STATEMENT OF PRIORITY

This application claims the benefit, under 35 U.S.C. §119(e), of U.S. Provisional Application Ser. No. 61/785,636, filed Mar. 14, 2013, the entire contents of which are incorporated by reference herein.

### STATEMENT OF GOVERNMENT SUPPORT

This invention was made with government support under Grant Nos. CA106523, CA95052, CA105055, CA133066 and CA135008 awarded by the National Institutes of Health and under Grant No. PC051264 awarded by the Department of Defense. The government has certain rights in the invention.

### FIELD OF THE INVENTION

The present invention provides methods and compositions directed to identification of genetic markers associated with prostate cancer.

### BACKGROUND OF THE INVENTION

Prostate cancer (PCa) is the most common cancer among men in the United States. Approximately 242,000 men are expected to be diagnosed with PCa in 2012. The lifetime probability of developing prostate cancer for men is 1 in 6 in the United States (US), the highest in comparison to other cancers. Although many PCa patients have an indolent (non-aggressive) form of the disease that may not even require treatment, a large number of men (˜28,000) die from this disease annually in the US alone. The inability to reliably distinguish between these two forms of the disease, especially at early stages, has resulted in over-treatment of many and under treatment of some. Therefore, it is critical to identify markers that can distinguish these two types of PCa patients at the time of diagnosis, as well as genes that drive cancer progression. Early identification of patients at either high- or low-risk for lethal disease has important clinical and public health implications. Such identification would enable clinically meaningful treatments at potentially curable stages for high-risk patients, while reducing unnecessary treatments for low-risk patients, thus reducing mortality and improving quality of life.

Clinicopathologic parameters can predict PCa biochemical recurrence, which while prognostic, is an imperfect surrogate of PCa-mortality. In addition, a high Gleason score, advanced tumor stage, and short PSA doubling time have been shown to be predictive of death from PCa. While these parameters are useful for the identification of patients at high risk of dying from PCa, they have limited utility in predicting mortality in patients with early stage disease when therapy is likely to be more effective. During the evolution of human cancers, genetic or molecular alterations that promote tumorigenesis precede traditional clinicopathologic changes that are associated with more aggressive disease. Thus, the elucidation of molecular makers that correlate with PCa-specific death may help identify a subset of patients with early stage but particularly aggressive cancers. Such patients would be candidates for early and perhaps more intense therapy. Moreover, the identification of biomarkers linked to PCa prognosis will also shed light on the mechanisms that drive the malignant phenotype that underlies PCa-mortality.

Although clinicopathologic parameters are commonly used predictors of outcome, they are insufficient for identification of potentially life-threatening forms of PCa prior to the development of advanced pathological phenotypes. Results from many studies suggest that most men with PSA-detected PCa have disease that will not progress to threaten their life. Extensive efforts have been made by many groups to improve upon the ability of current clinicopathologic parameters to predict aggressive forms of PCa using markers in somatic tissues. Although many have shown promise, none have been sufficiently validated and/or robust to justify their clinical application.

The present invention overcomes previous shortcomings in the art by identifying significant statistical associations between genetic markers and prostate cancer. Thus, the present invention provides methods and compositions for identifying a subject at increased risk of developing aggressive prostate cancer by detecting the genetic markers of this invention in the subject.

### SUMMARY OF THE INVENTION

In one aspect, the present invention provides a method of identifying a human subject as having an increased risk of having or developing aggressive prostate cancer, comprising detecting in a nucleic acid sample from the subject 1) a deletion at 1q42.2 from chr1:229894700-230947362 bp, 2) a deletion at 242.1 from chr2:139707778-140858852 bp, 3) a deletion at 1143 from chr11:113321588-113946501 bp, 4) an amplification at 141.3 from chr1:152725557-153275233 bp, or 5) any combination of 1-4 above, wherein the detection of same identifies the subject as having an increased risk of having or developing aggressive prostate cancer.

In an additional aspect, the present invention provides a method of identifying a human subject as having an increased likelihood of having or developing prostate cancer, comprising detecting in a nucleic acid sample from the subject 1) a deletion at 1q42.2 from chr1:229894700-230947362 bp, 2) a deletion at 242.1 from chr2:139707778-140858852 bp, 3) a deletion at 11q23 from chr11:113321588-113946501 bp, 4) an amplification at 1q21.3 from chr1:152725557-153275233 bp, or 5) any combination of 1-4 above, wherein the detection of same identifies the subject as having an increased likelihood of having or developing prostate cancer.

A further aspect of the present invention is a method of identifying a human subject as having an increased risk of having or developing aggressive prostate cancer, comprising detecting in a nucleic acid sample from the subject 1) a copy number alteration at 8q24.21 from chr8:128095593-129190507 bp (based on the physical position of the updated UCSC Genome Browser on Human Mar. 2006 (NCBI36/hg18) or from the corresponding physical positions defined in other versions or forms of the human genome browsers), 2) a copy number alteration at 1q21.3 from chr1:152725557-153275233 bp, 3) a copy number alteration at 1q21.33-22.1 from chr18:58288577-60834535 bp, 4) a copy number alteration at 8q21.13 from chr8:81128386-81867950 bp, 5) a copy number alteration at16q24.1 from chr16:82877051-83540927 bp, 6) a copy number alteration at10q23.31 from chr10:89613175-89888562 bp, 7) a copy number alteration at 17p13.1 from chr17:7501561-7781403 bp, and 8) any combination thereof, wherein the detection of same identifies the subject as having an increased risk of having) or developing aggressive prostate cancer.

An additional aspect of this invention is a method of identifying a human subject as having an increased likelihood of prostate cancer-specific death, comprising detecting in a nucleic acid sample from the subject 1) a copy number alteration at 8q24.21 from chr8:128095593-129190507 bp, 2) a copy number alteration at 1q21.3 from chr1:152725557-153275233 bp, 3) a copy number alteration at 18q21.33-22.1 from chr18:58288577-60834535 bp, 4) a copy number alteration at 8q21.13 from chr8:81128386-81867950 bp, 5) a copy number alteration at16q24.1 from chr16:82877051-83540927 bp, 6) a copy number alteration at10q23.31 from chr10:89613175-89888562 bp, 7) a copy number alteration at 17p13.1 from chr17:7501561-7781403 bp, and 8) any combination thereof, wherein the detection of same identifies the subject as having an increased likelihood of prostate cancer-specific death.

In yet further aspects, the present invention provides a method of identifying a human subject as having an increased risk of developing aggressive prostate cancer, comprising detecting in a nucleic acid sample from the subject 1) a copy number alteration in the MYC gene, 2) a copy number alteration in the ADAR gene, 3) a copy number alteration in the SERPIN5 gene, 4) a copy number alteration in the TPD52 gene, 5) a copy number alteration in the USP10 gene, 6) a copy number alteration in the PTEN gene, 7) a copy number alteration the TP53 gene, and 8) any combination thereof, wherein the detection of same identifies the subject as having an increased risk of developing aggressive prostate cancer.

As an additional aspect, the present invention provides a method of identifying a human subject as having an increased likelihood of prostate cancer-specific death, comprising detecting in a nucleic acid sample from the subject 1) a copy number alteration in the MYC gene, 2) a copy number alteration in the ADAR gene, 3) a copy number alteration in the SERPIN5 gene, 4) a copy number alteration in the TPD52 gene, 5) a copy number alteration in the USP10 gene, 6) a copy number alteration in the PTEN gene, 7) a copy number alteration the TP53 gene, and 8) any combination thereof, wherein the detection of same identifies the subject as having an increased likelihood of prostate cancer-specific death.

In addition, the present invention provides a method of identifying a human subject as having an increased risk of developing aggressive prostate cancer, comprising detecting in a nucleic acid sample from the subject a deletion in the gene PTEN and amplification of the gene MYC.

The present invention also provides a method of identifying a human subject as having an increased likelihood of prostate cancer-specific death, comprising detecting in a nucleic acid sample from the subject a deletion in the gene PTEN and amplification of the gene MYC.

Furthermore, the present invention provides a kit containing probes and other reagents for detecting a genetic marker (e.g., copy number alteration) of this invention.

Additionally provide herein is a computer-assisted method of identifying a proposed treatment and/or management for aggressive prostate cancer as an effective and/or appropriate treatment and/or management for a subject carrying a genetic marker correlated with aggressive prostate cancer, comprising the steps of (a) storing a database of biological data for a plurality of subjects, the biological data that is being stored including for each of said plurality of subjects: (i) a treatment type, (ii) at least one genetic marker associated with aggressive prostate cancer, and (iii) at least one disease progression measure for prostate cancer from which treatment efficacy can be determined; and then (b) querying the database to determine the dependence on said genetic marker of the effectiveness of a treatment type in treating prostate cancer, thereby identifying a proposed treatment as an effective and/or appropriate treatment for a subject carrying a genetic marker correlated with prostate cancer.

### BRIEF DESCRIPTION OF THE DRAWINGS

FIG. 1. Genomic identification of significant targets in cancer (GISTIC) using segmented DNA copy number data from the tumor genomes in a cohort from Johns Hopkins Hospital (JHH) in the U.S. Using a q-value of 0.01, a join segment size of 80, and a threshold for copy number amplification/deletion of 0.12, 20 significant regions were identified, including 15 deletions (left panel) and five amplifications (right panel). Left and right Y axes represent cytoband. Vertical line depicts FDR of 0.01. Top and bottom X axes represent G-score and q-value, respectively. * Novel regions and genes identified; ‡ refined regions with new genes identified; † known regions and genes confirmed in this study.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

The present invention is based on the unexpected discovery of genetic markers that are statistically associated with an increased risk of developing aggressive prostate cancer and an increased likelihood of prostate cancer-specific death. There are numerous benefits to carrying out the methods of this invention to identify a subject having an increased risk of developing aggressive prostate cancer, including but not limited to, identifying subjects who are good candidates for prophylactic and/or therapeutic treatment, and screening for cancer at an earlier time or more frequently than might otherwise be indicated, to increase the chances of early detection of an aggressive prostate cancer and reduce the incidence of prostate cancer-specific death.

Thus, in one aspect, the present invention provides a method of identifying a human subject as having an increased risk of having or developing aggressive prostate cancer, comprising detecting in a nucleic acid sample from the subject 1) a deletion at 1q42.2 from chr1:229894700-230947362 bp, 2) a deletion at 2q22.1 from chr2:139707778-140858852 bp, 3) a deletion at 11q23 from chr11:113321588-113946501 bp, 4) an amplification at 1q21.3 from chr1:152725557-153275233 bp, or 5) any combination of 1-4 above, wherein the detection of same identifies the subject as having an increased risk of having or developing aggressive prostate cancer.

In an additional aspect, the present invention provides a method of identifying a human subject as having an increased likelihood of prostate cancer-specific death, comprising detecting in a nucleic acid sample from the subject 1) a deletion at 1q42.2 from chr1:229894700-230947362 bp, 2) a deletion at 2q22.1 from chr2:139707778-140858852 bp, 3) a deletion at 11q23 from chr1:113321588-113946501 bp, 4) an amplification at 1q21.3 from chr1:152725557-153275233 bp, or 5) any combination of 1-4 above, wherein the detection of same identifies the subject as having an increased likelihood of prostate cancer-specific death.

A further aspect of the present invention is a method of identifying a human subject as having an increased risk of developing aggressive prostate cancer, comprising detecting in a nucleic acid sample from the subject 1) a copy number alteration at 8q24.21 from chr8:128095593-129190507 bp, 2) a copy number alteration at 1q21.3 from chr1:152725557-153275233 bp, 3) a copy number alteration at 18q21.33-22.1 from chr18:58288577-60834535 bp, 4) a copy number alteration at 8q21.13 from chr8:81128386-81867950 bp, 5) a copy number alteration at16q24.1 from chr16:82877051-83540927 bp, 6) a copy number alteration at10q23.31 from chr10:89613175-89888562 bp, 7) a copy number alteration at 17p13.1 from chr17:7501561-7781403 bp, and 8) any combination thereof, wherein the detection of same identifies the subject as having an increased risk of developing aggressive prostate cancer. An additional aspect of this invention is a method of identifying a human subject as having an increased likelihood of prostate cancer-specific death, comprising detecting in a nucleic acid sample from the subject 1) a copy number alteration at 8q24.21 from chr8:128095593-129190507 bp, 2) a copy number alteration at 1q21.3 from chr1:152725557-153275233 bp, 3) a copy number alteration at 18q21.33-22.1 from chr18:58288577-60834535 bp, 4) a copy number alteration at 8q21.13 from chr8:81128386-81867950 bp, 5) a copy number alteration at16q24.1 from chr16:82877051-83540927 bp, 6) a copy number alteration at10q23.31 from chr10:89613175-89888562 bp, 7) a copy number alteration at 17p13.1 from chr17:7501561-7781403 bp, and 8) any combination thereof, wherein the detection of same identifies the subject as having an increased likelihood of prostate cancer-specific death.

In yet further aspects, the present invention provides a method of identifying a human subject as having an increased risk of developing aggressive prostate cancer, comprising detecting in a nucleic acid sample from the subject 1) a copy number alteration in the MYC gene, 2) a copy number alteration in the ADAR gene, 3) a copy number alteration in the SERPIN5 gene, 4) a copy number alteration in the TPD52 gene, 5) a copy number alteration in the USP10 gene, 6) a copy number alteration in the PTEN gene, 7) a copy number alteration the TP53 gene, and 8) any combination thereof, wherein the detection of same identifies the subject as having an increased risk of developing aggressive prostate cancer.

As an additional aspect, the present invention provides a method of identifying a human) subject as having an increased likelihood of prostate cancer-specific death, comprising detecting in a nucleic acid sample from the subject 1) a copy number alteration in the MYC gene, 2) a copy number alteration in the ADAR gene, 3) a copy number alteration in the SERPIN5 gene, 4) a copy number alteration in the TPD52 gene, 5) a copy number alteration in the USP10 gene, 6) a copy number alteration in the PTEN gene, 7) a copy number alteration the TP53 gene, and 8) any combination thereof, wherein the detection of same identifies the subject as having an increased likelihood of prostate cancer-specific death.

In addition, the present invention provides a method of identifying a human subject as having an increased risk of developing aggressive prostate cancer, comprising detecting in a nucleic acid sample from the subject a deletion in the gene PTEN and amplification of the gene MYC.

Also provided in the present invention is a method of identifying a human subject as having an increased likelihood of prostate cancer-specific death, comprising detecting in a nucleic acid sample from the subject a deletion in the gene PTEN and amplification of the gene MYC.

Regarding a deletion in the gene PTEN, both hemizygous deletion (one of the two alleles or copies was deleted, about 27% of PCa patients) and homozygous deletion (both alleles/copies were deleted, about 13% of PCa patients) were observed in the tumors of the patients with prostate cancer. About 90% of hemizygous deletions affected the whole gene of PTEN, with about 10% affecting only a part of PTEN. About 60% of the homozygous deletions affected the whole gene of PTEN, while 40% of homozygous deletions affected only a part the gene.

When a subject is identified as having an increased risk of developing aggressive prostate cancer, various steps can be taken. For example, the methods of this invention could be used for each PCa-positive biopsy core to determine whether it contains the seven CNAs described herein that are associated with lethal PCa. If any core from a subject has the CNA signature at any or combination of these seven genes, the subject will be more likely to have a poor outcome. Therefore, a physician may choose to treat the subject aggressively at critical times using surgery, radiation, hormonal therapy and/or chemotherapy. If a subject does have CNAs of these seven genes, a physician may manage the disease through active surveillance. If a PTEN deletion is detected in the tumor of a subject, a physician may add PI3K pathway inhibitors as part of the treatment strategy (e.g., PI3K-Akt-mTOR pathway treatment to target PTEN deletion). If the) subject harbors a TP53 deletion in the tumor, a physician may choose gene therapy to restore p53, and/or another drug or drugs to activate the p53 pathway.

Thus, as a nonlimiting example, the methods of this invention can be used to guide a subject's prostate cancer treatment regimen, comprising carrying out any of the methods of this invention and guiding the subject's treatment regimen such that detection of the CNA signature at any or a combination of the genes in the seven genomic regions associated with lethal prostate cancer described herein in a subject with prostate cancer leads to more active surveillance and/or more aggressive treatment and/or management of the subject than would be implemented for a subject with prostate cancer in whom none of these markers were detected, including surgery, radiation therapy, hormone therapy and/or chemotherapy as well as more frequent timing and duration of such therapies, and no detection of these genetic markers in a subject with prostate cancer leads to standard treatment and/or routine monitoring as are well known in the art.

As another nonlimiting example, the methods of this invention can be used to guide a physician's actions with regard to a subject in whom prostate cancer has not been diagnosed or detected, comprising carrying out any of the methods of this invention and guiding the physician's actions such that detection of the CNA signature at any or a combination of the genes in the seven genomic regions described herein in a subject without prostate cancer leads to more active surveillance and/or more aggressive prophylactic treatment of the subject than would be implemented for a subject without prostate cancer in whom none of these markers were detected, including surgery, radiation therapy, hormone therapy and/or chemotherapy as well as more frequent timing and duration of such therapies, and no detection of these genetic markers in a subject without prostate cancer leads to standard prophylactic treatment and/or routine monitoring as are well known in the art.

The present invention relates to a set of genomic regions with DNA copy number alterations or abnormalities (CNAs) for identifying aggressive prostate cancers (PCa) leading to cancer specific mortality, a set of genomic regions in which CNAs are not or rarely observed in cancer cells for using as internal references for calculating and defining CNAs and methods of using these described genomic regions for identifying aggressive PCa imposing higher risk to the patients dying from this disease at early stage.

In the present invention, the identification of somatic DNA CNAs in the tumor genome that predict for PCa-specific death after prostatectomy for clinically localized disease is described. Using a retrospective study consisting of four cohorts of PCa patients with distinct clinicopathologic profiles from different geographical locations, the identification and validation of CNAs that are significantly associated with PCa-mortality is demonstrated, with some of them being independent of Gleason grade, pathological stage, and pre-operative PSA levels. Furthermore, 69 genomic regions in which CNAs are not or rarely observed in the tumor cells are defined for use as references in testing CNAs in PCa via various methods. Methods of using these described genomic regions for identifying aggressive PCa imposing higher risk to the patients dying from this disease are also included herein.

A set of genomic regions with DNA copy number alterations (CNAs) for identifying significant targets in prostate cancer according to embodiments of present invention may include deletions (Table 7 and Table 8) or amplifications (Table 9 and Table 10) of one or more genes, with the number of regions and genes dependent upon the criteria q-value and join segment size.

Identification of the chromosome regions described herein is based on the UCSC Genome Browser on Human Mar. 2006 (NCBI36/hg18) Assembly.

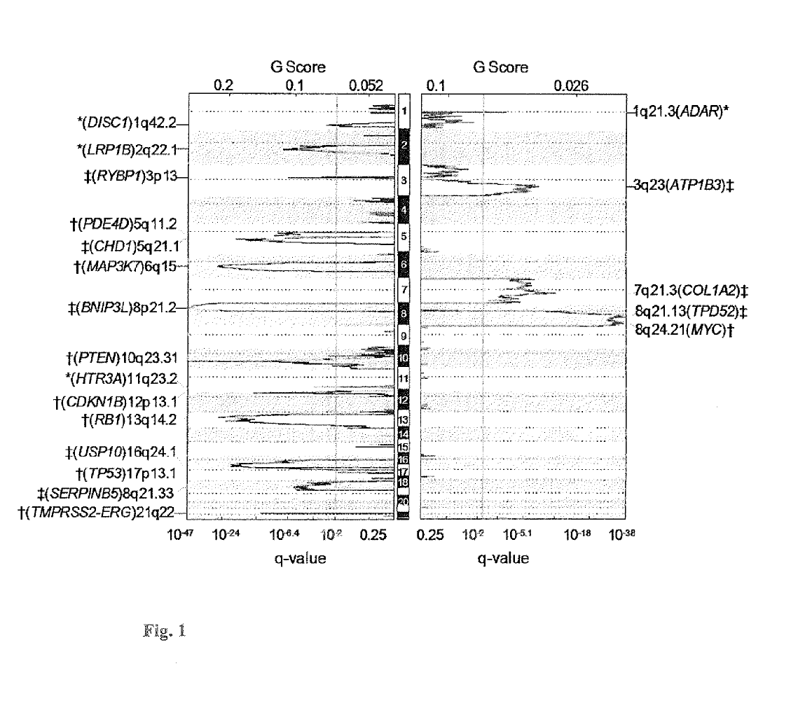

In some embodiments, a q-value of 0.25 and a joint segment size of 60 probes can be used for selection of significant cancer targets among the CNAs. In some embodiments, a q-value of 0.01 and a joint segment size of 80 probes can be used for selection of significant cancer targets among the CNAs. Using the SNP arrays and GISTIC algorithm with a q-value of 0.01 to analyze 125 primary tumors from the JHH discovery cohort, the 20 most significant CNAs along with the most commonly gained or deleted gene(s) within each region are identified (FIG. 1, Table 8 and Table 10). Fifteen of the CNAs represent chromosomal deletions and five are gains or amplification.

The locations of the significant targets are defined by the cytobands, while the size of each region may be defined by the wide peak boundaries. As the number of genes in each of the significant regions varies, the CNAs may be named by the known or suspected tumor suppressor or oncogene within the altered sequences or by the first gene listed by GISTIC in the region.

A set of genomic regions with DNA CNAs for identifying the aggressive PCa leading to higher risk of cancer mortality according to some embodiments of the present invention may includes one or more genes; with a P value<0.05 resulted from a univariate analysis (Table 2). These include regions at 8q24.21 from chr8:128095593-129190507 base pair (bp), 1q21.3 from chr1:152725557-153275233 bp, 18q21.33-22.1 from chr18:58288577-60834535 bp, 8q21.13 D from chr8:81128386-81867950 bp, 16q24.1 from chr16:82877051-83540927 bp, 10q23.31 from chr10:89613175-89888562 bp, 17p13.1 from chr17:7501561-7781403 bp, including but not limited to the genes MYC, ADAR, SERPINB5, TPD52, USP10, PTEN, and TP53.

A set of genomic regions with DNA CNAs contributing additional prognostic mortality-information independent of that provided by pathologic stage, Gleason score, and initial PSA level, according to some embodiments of the present invention may be determined by multivariate analysis and therefore, includes deletion of the sequences at 10q23.31 and/or amplification of the sequences at 8q24.21, represented by the genes PTEN and MYC, respectively (Table 3).

A joint effect of alterations at PTEN and MYC on PCa-specific mortality may be explored to identify patients who may have even higher risk of having aggressive PCa leading to cancer specific death (Table 4).

A set of genomic regions in which CNAs are not or rarely observed in the tumor cells for use as references for calculating and defining CNAs according to embodiments of the present invention may include one or more or any combination of the sequences described in Table 11.

The methods for detecting DNA CNAs in identification of patients with aggressive PCa leading to high risk of cancer specific death may include comparative genomic hybridization or the same, such as metaphase (or conventional) and BAC/oligo/cDNA/single nucleotide polymorphic (SNP; or array-based) hybridization with various resolutions. It is preferable to use fluorescent in situ hybridization for detection of CNAs in clinical settings for identification of patients with aggressive PCa leading to high risk of dying. It is even more preferable to use a PCR based method, including but not limited to quantitative PCR and multiplex ligation-dependent probe amplification, for analyzing CNAs at the specific regions depicted in the present invention.

The sources of DNA for detecting CNAs in identification of patients with aggressive PCa leading to high risk of cancer specific death may include biological fluids including but not limited to blood, serum/plasma and urine, circulating tumor cells (CTCs), and tumor as well as matched normal tissues from the prostate and other anatomical sites of metastases. In some embodiments, DNA derived from blood and serum/plasma can be used. In some embodiments, DNA isolated from formalin-fixed, paraffin-embedded tissues can be used. In some) embodiments, DNA from fresh frozen, including but not limited to CTCs, biopsy and prostatectomy, tissues can be used.

In some embodiments, methods other than CGH based, such as PCR based methods can be used to analyze DNA derived from tissues or sources obtained via less invasive approaches than prostatectomy.

Methods and algorithms will be used for identification of patients with aggressive PCa that may lead to early cancer specific death if not treated aggressively or appropriately. The current invention may also be used in an active surveillance program to monitor the progression of PCa. In addition it may be used to monitor the response to treatments of PCa.

Various detection protocols can be used in the methods of this invention to detect the CNAs described herein. Nonlimiting examples include comparative genomic hybridization such as metaphase (or conventional) and BAC/oligo/cDNA/single nucleotide polymorphic (SNP; or array-based) hybridization with various resolutions; Affymetrix SNP array, NanoString technology (such as nCounter), multiplex ligation-dependent probe amplification (MLPA); fluorescence in situ hybridization (FISH); an amplification reaction (e.g., quantitative polymerase chain reaction (PCR)); an amplification reaction and single base extension (e.g., wherein the single base extension is spotted on a silicone chip); matrix-assisted laser desorption/ionization-time of flight mass spectrometry (MALDI-TOF-MS); sequencing, hybridization; restriction endonuclease digestion analysis; electrophoresis; or any combination thereof.

In particular embodiments, detection can be carried out by multiplex ligation-dependent probe amplification (MLPA). The use of MLPA has the advantage that all seven regions can be measured simultaneously in an MLPA kit and fresh frozen and formalin fixed paraffin-embedded samples can be used as a source of DNA for analysis. Such a MLPA-based method can be used to cost effectively measure all seven DNA copy number alterations in prostate biopsy tissues. Some strengths of this method are: 1) all seven regions associated with lethal prostate cancer are included, 2) at least 3 probes for each of seven targeted regions, 3) no known SNPs within the probes, and 4) all internal reference regions do not or rarely have known copy number alterations in the prostate tumors tested.

Thus in some aspects, the present invention provides a kit for carrying out the methods of this invention (e.g., a kit comprising reagents, as well as a probe mix, to detect the CNAs of this invention in a nucleic acid sample). Such a kit can comprise oligonucleotides (e.g., primers, probes, primer/probe sets, etc.), reagents, buffers, etc., as would be known in the art, for the detection of the genetic markers of this invention in a nucleic acid sample. Such oligonucleotides can be identified and prepared and employed in methods according to the teachings and protocols described herein and as are well known in the art. A kit of this invention can further comprise blocking probes, labeling reagents, blocking agents, restriction enzymes, antibodies, sampling devices, positive and negative controls, etc., as would be well known to those of ordinary skill in the art.

### DEFINITIONS

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the term “prostate cancer” or “PCa” describes an uncontrolled (malignant) growth of cells in the prostate gland, which is located at the base of the urinary bladder and is responsible for helping control urination as well as forming part of the semen. Symptoms of prostate cancer can include, but are not limited to, urinary problems (e.g., not being able to urinate; having a hard time starting or stopping the urine flow; needing to urinate often, especially at night; weak flow of urine; urine flow that starts and stops; pain or burning during urination), difficulty having an erection, blood in the urine and/or semen, and/or frequent pain in the lower back, hips, and/or upper thighs.

As used herein, the term “aggressive prostate cancer” means prostate cancer that is poorly differentiated, having a Gleason grade of 7 or above. An “indolent prostate cancer” means prostate cancer having a Gleason grade below 7 (e.g., 6 or less). The Gleason grading system is the most commonly used method for grading PCa and is well known in the art.

The term “chromosome region” as used herein refers to a part of a chromosome defined) either by anatomical details, especially by banding, or by its linkage groups.

Also as used herein, “linked” describes a region of a chromosome that is shared more frequently in family members or members of a population manifesting a particular phenotype and/or affected by a particular disease or disorder, than would be expected or observed by chance, thereby indicating that the gene or genes or other identified marker(s) within the linked chromosome region contain or are associated with an allele that is correlated with the phenotype and/or presence of a disease or disorder (e.g., aggressive PCa), or with an increased or decreased likelihood of the phenotype and/or of the disease or disorder. Once linkage is established, association studies (linkage disequilibrium) can be used to narrow the region of interest or to identify the marker (e.g., allele or haplotype) correlated with the phenotype and/or disease or disorder.

Furthermore, as used herein, the term “linkage disequilibrium” or “LD” refers to the occurrence in a population of two or more (e.g., 2, 3, 4, 5, 6, 7, 8, 9, 10, etc.) linked alleles at a frequency higher or lower than expected on the basis of the gene frequencies of the individual genes. Thus, linkage disequilibrium describes a situation where alleles occur together more often than can be accounted for by chance, which indicates that the two or more alleles are physically close on a DNA strand.

The term “genetic marker” or “polymorphism” as used herein refers to a characteristic of a nucleotide sequence (e.g., in a chromosome) that is identifiable due to its variability among different subjects (i.e., the genetic marker or polymorphism can be a single nucleotide polymorphism, an allele of a single nucleotide polymorphism, a restriction fragment length polymorphism, a microsatellite, a deletion of nucleotides, an addition of nucleotides, a substitution of nucleotides, a repeat or duplication of nucleotides, a translocation of nucleotides, a copy number alteration, and/or an aberrant or alternate splice site resulting in production of a truncated or extended form of a protein, etc., as would be well known to one of ordinary skill in the art).

A “single nucleotide polymorphism” (SNP) in a nucleotide sequence is a genetic marker that is polymorphic for two (or in some case three or four) alleles. SNPs can be present within a coding sequence of a gene, within noncoding regions of a gene and/or in an intergenic (e.g., intron) region of a gene. A SNP in a coding region in which both forms lead to the same polypeptide sequence is termed synonymous (i.e., a silent mutation) and if a different polypeptide sequence is produced, the alleles of that SNP are non-synonymous. SNPs that are not in protein coding regions can still have effects on gene splicing, transcription factor binding and/or the sequence of non-coding RNA.

The SNP nomenclature provided herein refers to the official Reference SNP (rs) identification number as assigned to each unique SNP by the National Center for Biotechnological Information (NCBI), which is available in the GenBank® database.

In some embodiments, the term genetic marker is also intended to describe a phenotypic effect of an allele or haplotype, including for example, an increased or decreased amount of a messenger RNA, an increased or decreased amount of protein, an increase or decrease in the copy number of a gene, production of a defective protein, tissue or organ, etc., as would be well known to one of ordinary skill in the art.

An “allele” as used herein refers to one of two or more alternative forms of a nucleotide sequence at a given position (locus) on a chromosome (e.g., at a single nucleotide polymorphism). An allele can be a nucleotide present in a nucleotide sequence that makes up the coding sequence of a gene and/or an allele can be a nucleotide in a non-coding region of a gene (e.g., in a genomic sequence). A subject's genotype for a given gene is the set of alleles the subject happens to possess. As noted herein, an individual can be heterozygous or homozygous for any allele of this invention.

Also as used herein, a “haplotype” is a set of alleles on a single chromatid that are statistically associated. It is thought that these associations, and the identification of a few alleles of a haplotype block, can unambiguously identify all other alleles in its region. The term “haplotype” is also commonly used to describe the genetic constitution of individuals with respect to one member of a pair of allelic genes; sets of single alleles or closely linked genes that tend to be inherited together.

The terms “increased risk” and “decreased risk” as used herein define the level of risk that a subject has of developing aggressive prostate cancer, as compared to a control subject that does not have the alleles of this invention in the control subject's nucleic acid.

A sample of this invention can be any sample containing nucleic acid from a subject, as would be well known to one of ordinary skill in the art. Nonlimiting examples of a sample of this invention include a cell, a body fluid, a tissue, biopsy or surgery material, a washing, a swabbing, etc., as would be well known in the art.

A subject of this invention is any animal that is susceptible to prostate cancer as defined herein and can include, for example, humans, as well as animal models of prostate cancer (e.g., rats, mice, dogs, nonhuman primates, etc.). In some aspects of this invention, the subject can be Caucasian (e.g., white; European-American; Hispanic), as well as of black African ancestry (e.g., black; African, Sub-Saharan African, African American; African-European; African-Caribbean, etc.) or Asian. In further aspects of this invention, the subject can have a family history of prostate cancer or aggressive prostate cancer (e.g., having at least one first degree relative having or diagnosed with prostate cancer or aggressive prostate cancer) and in some embodiments, the subject does not have a family history of prostate cancer or aggressive prostate cancer. Additionally a subject of this invention can have a diagnosis of prostate cancer or aggressive prostate cancer in certain embodiments and in other embodiments, a subject of this invention does not have a diagnosis of prostate cancer or aggressive prostate cancer. In yet further embodiments, the subject of this invention can have an elevated prostate-specific antigen (PSA) level and in other embodiments, the subject of this invention can have a normal or non-elevated PSA level. In some embodiments, the PSA level of the subject may not be known and/or has not been measured.

As used herein, “nucleic acid” encompasses both RNA and DNA, including cDNA, genomic DNA, mRNA, synthetic (e.g., chemically synthesized) DNA and chimeras, fusions and/or hybrids of RNA and DNA. The nucleic acid can be double-stranded or single-stranded. Where single-stranded, the nucleic acid can be a sense strand or an antisense strand. In some embodiments, the nucleic acid can be synthesized using oligonucleotide analogs or derivatives (e.g., inosine or phosphorothioate nucleotides, etc.). Such oligonucleotides can be used, for example, to prepare nucleic acids that have altered base-pairing abilities or increased resistance to nucleases.

An “isolated nucleic acid” is a nucleotide sequence that is not immediately contiguous with nucleotide sequences with which it is immediately contiguous (one on the 5′ end and one on the 3′ end) in the naturally occurring genome of the organism from which it is derived or in which it is detected or identified. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (e.g., when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about five nucleotides to about 500 nucleotides (e.g. 5, 6, 7, 8, 9, 10, 12, 15, 18, 20, 21, 22, 25, 30, 35, 40, 45, 50, 55, 60, 65, 70, 75, 80, 85, 90, 100, 125, 150, 175, 200, 250, 300, 350, 400, 450, 500, 550 or 600 nucleotides). In some embodiments, for example, an oligonucleotide can be from about 15 nucleotides to about 30 nucleotides, or about 20 nucleotides to about 25 nucleotides, which can be used, for example, as a primer in a polymerase chain reaction (PCR) amplification assay and/or as a probe in a hybridization assay or in a microarray. Oligonucleotides of this invention can be natural or synthetic, e.g., DNA, RNA, PNA, LNA, modified backbones, etc., as are well known in the art.

The present invention further provides fragments of the nucleic acids of this invention, which can be used, for example, as oligonucleotides, primers and/or probes. Such fragments or oligonucleotides can be detectably labeled, ligated or modified, for example, to include and/or incorporate a restriction enzyme cleavage site when employed as a primer in an amplification (e.g., PCR) assay.

The detection of a polymorphism, genetic marker or allele of this invention can be carried out according to various protocols standard in the art and as described herein for analyzing nucleic acid samples and nucleotide sequences, as well as identifying specific nucleotides in a nucleotide sequence.

For example, nucleic acid can be obtained from any suitable sample from the subject that will contain nucleic acid and the nucleic acid can then be prepared and analyzed according to) well-established protocols for the presence of genetic markers according to the methods of this invention. In some embodiments, analysis of the nucleic acid can be carried by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides for use as primers and/or probes for detecting and/or identifying genetic markers according to the methods of this invention.

In some embodiments of this invention, detection of an allele or combination of alleles of this invention can be carried out by an amplification reaction and single base extension. In particular embodiments, the product of the amplification reaction and single base extension is spotted on a silicone chip.

In yet additional embodiments, detection of an allele or combination of alleles of this invention can be carried out by matrix-assisted laser desorption/ionization-time of flight mass spectrometry (MALDI-TOF-MS).

It is further contemplated that the detection of an allele or combination of alleles of this invention can be carried out by various methods that are well known in the art, including, but not limited to nucleic acid sequencing, hybridization assay, restriction endonuclease digestion analysis, ligation, electrophoresis, and any combination thereof.

The genetic markers (e.g., CNAs) of this invention are correlated with (i.e., identified to be statistically associated with) aggressive prostate cancer as described herein according to methods well known in the art and as disclosed in the Examples provided herein for statistically correlating genetic markers with various phenotypic traits, including disease states and pathological conditions as well as determining levels of risk associated with developing a particular phenotype, such as a disease or pathological condition. In general, identifying such correlation involves conducting analyses that establish a statistically significant association and/or a statistically significant correlation between the presence of a genetic marker or a combination of markers and the phenotypic trait in a population of subjects and controls (e.g., a population of subjects in whom the phenotype is not present or has not been detected). The correlation can involve one or more than one genetic marker of this invention (e.g., two, three, four, five, or more) in any combination. An analysis that identifies a statistical association (e.g., a significant association) between the marker or combination of markers and the phenotype establishes a correlation between the presence of the marker or combination of markers in a population of subjects and the particular phenotype being analyzed. A level of risk (e.g., increased or decreased) can then be determined for an individual on the basis of such population-based analyses.

Thus, in certain embodiments, the present invention provides a method of screening a subject for a genetic marker (e.g., a copy number alteration) that is associated with aggressive prostate cancer, comprising: a) performing a population based study to detect genetic markers (e.g., copy number alterations) in a group of subjects with aggressive prostate cancer and a group of control subjects; b) identifying copy number alterations in the aggressive prostate cancer group of subjects that are statistically associated with the presence of aggressive prostate cancer; and c) screening a subject for the presence of the copy number alterations identified in step (b).

The present invention further provides a method of identifying an effective and/or appropriate (i.e., for a given subject's particular condition or status) treatment regimen for a subject with aggressive prostate cancer, comprising detecting one or more of the genetic markers associated with aggressive prostate cancer of this invention in the subject, wherein the one or more genetic markers are further statistically correlated with an effective and/or appropriate treatment regimen for aggressive prostate cancer according to protocols as described herein and as are well known in the art.

Also provided is a method of identifying an effective and/or appropriate treatment regimen for a subject with aggressive prostate cancer, comprising: a) correlating the presence of one or more genetic markers of this invention in a test subject or population of test subjects with aggressive prostate cancer for whom an effective and/or appropriate treatment regimen has been identified; and b) detecting the one or more markers of step (a) in the subject, thereby identifying an effective and/or appropriate treatment regimen for the subject.

Further provided is a method of correlating a genetic marker of this invention with an effective and/or appropriate treatment regimen for aggressive prostate cancer, comprising: a) detecting in a subject or a population of subjects with aggressive prostate cancer and for whom an effective and/or appropriate treatment regimen has been identified, the presence of one or more genetic markers or polymorphisms of this invention; and b) correlating the presence of the one or more genetic markers of step (a) with an effective treatment regimen for aggressive prostate cancer.

Examples of treatment regimens for prostate cancer are well known in the art. Subjects who respond well to particular treatment protocols can be analyzed for specific genetic markers and a correlation can be established according to the methods provided herein. Alternatively, subjects who respond poorly to a particular treatment regimen can also be analyzed for particular genetic markers correlated with the poor response. Then, a subject who is a candidate for treatment for aggressive prostate cancer can be assessed for the presence of the appropriate genetic markers and the most effective and/or appropriate treatment regimen can be provided as early as possible.

In some embodiments, the methods of correlating genetic markers with treatment regimens of this invention can be carried out using a computer database. Thus the present invention provides a computer-assisted method of identifying a proposed treatment for aggressive prostate cancer and/or appropriate treatment for a subject carrying a genetic marker correlated with aggressive prostate cancer.

The method can include the steps of (a) storing a database of biological data for a plurality of subjects, the biological data that is being stored including for each of said plurality of subjects, for example, (i) a treatment type, (ii) at least one genetic marker associated with aggressive prostate cancer and (iii) at least one disease progression measure for aggressive prostate cancer from which treatment efficacy can be determined; and then (b) querying the database to determine the correlation between the presence of said genetic marker and the effectiveness of a treatment type in treating aggressive prostate cancer, to thereby identify a proposed treatment as an effective for aggressive prostate cancer and/or an appropriate treatment for a subject carrying a genetic marker correlated with aggressive prostate cancer.

In some embodiments, treatment information for a subject is entered into the database (through any suitable means such as a window or text interface), genetic marker information for that subject is entered into the database, and disease progression information is entered into the database. These steps are then repeated until the desired number of subjects has been entered into the database. The database can then be queried to determine whether a particular treatment is effective for subjects carrying a particular marker or combination of markers, not effective for subjects carrying a particular marker or combination of markers, etc. Such querying can be carried out prospectively or retrospectively on the database by any suitable means, but is generally done by statistical analysis in accordance with known techniques, as described herein.

The following examples are not intended to limit the scope of the claims to the invention, but are rather intended to be exemplary of certain embodiments. Any variations in the exemplified methods that occur to the skilled artisan are intended to fall within the scope of the present invention. As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

### EXAMPLES

### Example 1

Using DNA samples from frozen tumors of 125 patients treated by radical prostatectomy with a median follow-up of ˜seven years from Johns Hopkins Hospital (JHH) in the US and the algorithm of Genomic Identification of Significant Targets in Cancer (GISTIC), seven copy number alterations (CNAs) were identified that were significantly associated with early PCa-specific mortality. These include gains of chromosomal regions that contain the genes MYC, ADAR, or TPD52 and losses of sequences that incorporate SERPINB5, USP10, PTEN, or TP53. Furthermore, multivariate analysis revealed that deletion of the gene PTEN and amplification of the gene MYC contributed additional prognostic information independent of that provided by traditional clinicopathologic measurements, such as pathologic stage, Gleason score, and initial PSA level. Finally, 69 genomic regions in which CNAs were not or rarely observed in the tumor cells were defined using DNA copy number data from 5 different PCa cohorts for use as references in testing CNAs in PCa. The present invention describes the use of these CNAs for identification of patients with aggressive PCa that may lead to high risk of early cancer specific death if patients are not treated aggressively or appropriately. These CNAs allow for more accurate patient prognosis, at the time of surgery or biopsy, and help guide the selection of appropriate therapy and disease management strategy.

Genetic Markers Associated with Early Cancer-Specific Mortality Following Prostatectomy.

PCa is the most common cancer among men in the United States with ˜242,000 expected to be diagnosed in 2012.¹ Although many of these tumors may be indolent and not require treatment, ˜28,000 men die from this disease annually.¹ Treatment options for patients who present with localized disease include surgery, radiotherapy, medical therapies, or surveillance. The outcome following prostatectomy is generally excellent, with large series reporting PCa-specific mortality rates as ˜15% or less after ten years or more.^{2,3} However, the Swedish randomized trial showed that the benefit of surgery over conservative management was modest, with a projected PCa-specific mortality rate after fifteen years of 14.6% versus 20.7%, respectively.⁴ Although this study showed that prostatectomy can prevent cancer deaths, a sizable proportion of patients had tumors that were not life-threatening at least within the fifteen-year time frame. Conversely, one in seven of the patients who have a prostatectomy nonetheless relapses and dies of progressive cancer.

From this and other studies, it is apparent that the care of patients with early PCa could be improved by the development of novel therapies and tumor prognostic markers. These can help distinguish between those patients with life-threatening disease that require more aggressive therapy from those with indolent disease which could be treated conservatively. One strategy to achieve these goals is to exploit knowledge of the molecular pathogenesis of the disease. As in all cancers, the malignant phenotype of PCa is driven by the acquisition and collaboration of somatic alterations.⁵⁻⁸ Thus, analysis of copy number abnormalities in tumor genomes provides insights into the specific genes and molecular pathways that promote tumorigenesis and determine the clinical course. As described here, high resolution SNP arrays and GISTIC, respectively, were used to identify and determine the significant CNAs in tumors obtained at prostatectomy from 125 patients. These CNAs were then correlated with clinicopathologic features and clinical outcome. The results revealed new CNAs and genes that likely contribute to the pathogenesis of PCa and associations between specific CNAs and early PCa-specific mortality.

The initial discovery cohort consisted of 141 patients who underwent radical prostatectomy at Johns Hopkins Hospital (JHH) in the U.S. between 1988 and 2004. To minimize the effects from confounding factors and to include as many eligible patients as possible for better statistical power, the effects of clinical variables were evaluated, including Gleason score, metastasis, race and adjuvant therapy on PCa-specific mortality. The following patients were eliminated: 5 patients without Gleason score, 5 patients with metastases and 6 patients without death information from the original cohort of 141 patients. The resulting 125 patients were then used to analyze the effects of race and no association between race and PCa-specific death was found. Therefore 115 Caucasian, eight African Americans and two others were included in the study and race effects were not stratified in the analysis. In addition a significant correlation (P=0.04) was found between patients with higher Gleason scores and patients with adjuvant therapy, however this did not lead to a demonstrable reduction in PCa-mortality. Therefore, 36 patients who received hormone- and/or chemo-therapies were included in the final study population of 125 eligible patients with a median follow-up of seven years from JHH. As shown in Table 1, many of these patients had a more aggressive form of PCa; 34%, 33%, and 44% of patients, respectively had a pathologic Gleason score≧8, a pathologic stage≧T3b, and pretreatment serum PSA>10 ng/mL. Consistent with this frequency of high risk disease, 22 of these patients (˜18%) died of PCa, while the remaining 103 were still alive or died from other causes as of June, 2009. Eight out of 22 PCa-specific deaths happened within five years after surgery in the JHH cohort; the five-year survival rate was 64%.

The second cohort included 103 prostatectomy patients who were treated between 2002 and 2008 with a median follow-up of about five years from the Karolinska University Hospital (KUH) in Sweden (Table 1). In contrast to the JHH cohort described above, most of the Swedish patients had a less aggressive form of PCa. About 85%, 82% and 64% of patients, respectively had a pathologic Gleason score≦7, a pathologic stage<T3, and pretreatment serum PSA≦10 ng/mL. Consistent with this frequency of low risk disease and shorter follow-up time, only four subjects died from PCa, while the remaining 99 were still alive or died from other causes at the time of data analysis as of December, 2010. Three out of four PCa-specific deaths happened within five years after surgery in the KUH cohort; the five-year survival rate was 25%. A third cohort was composed of 216 patients from Memorial Sloan Kettering Cancer Center (MSKCC) with clinicopathologic and survival information publically available.⁷ An additional group of 14 JHH patients who died of progressive PCa and underwent autopsy provided tumor samples for the study of lethal PCa.⁶ Informed consent was obtained and the Institutional Review Board/Ethics Committee in participating institutions approved the study.

Somatic tumor and matched normal DNA from patients of the JHH and KUH cohorts was prepared and used for SNP array analysis of genome-wide CNAs as described previously.⁶ GISTIC method⁹ was used to identify significant CNAs. As the number of genes in each of the significant regions varied, the CNAs were named by the known or suspected tumor suppressor or oncogene within the altered sequences or by the first gene listed GISTIC⁹ in the region.

Association of clinicopathologic variables and DNA CNAs with PCa-specific mortality was explored using logistic regression. CNAs were coded as either deletions or gains. The primary outcome was mortality due to PCa. Both univariate and multivariate analyses were incorporated in the logistic regression model. First, univariate analysis was performed in order to check the relationship between the end point and the explanatory variables. Second, for associations with PCa-specific mortality, multiple variables were included in the logistic regression model based on stepwise model selection and all models retained clinicopathologic variables, including age, Gleason score, and preoperative PSA, whether they were significant. Tumor-stage was not included the because of multicollinearity with Gleason score. Studies were also done to test for any pair of alterations that was significantly associated with PCa-specific mortality in a multivariate analysis. The analysis was restricted to significant alterations because of the small sample size. The P value for the difference between the two areas under the receiver operating characteristic curve (AUC) was calculated using a nonparametric approach.¹⁰

To explore the joint effect of alterations at PTEN and MYC on PCa-specific mortality, the raw data were displayed using a four by two table. Odds ratio (OR) and the 95% confidence intervals were calculated based on the contingency table. P-values were calculated using Fisher's exact test, the Cochran-Armitage trend test, and Cochran-Mantel-Haenszel (CMH) test. All of the analyses were performed using SAS 9.2 Software.

Genome-Wide Analysis of Tumor DNA Reveals the 20 Most Significant CNAs in Clinically Localized PCa.

Using the SNP arrays and GISTIC algorithm with a q-value of 0.01 to analyze 125 primary tumors from the JHH discovery cohort, the 20 most significant CNAs were identified, along with the most commonly gained or deleted gene(s) within each region (FIG. 1). Fifteen of the CNAs represented chromosomal deletions and five were gains or amplification.

Four of the CNAs, consisting of deletions that involved DISC1 (102.2), LRP1B (2q22.1) and HRT3A (11q23) or gains of ADAR1 (1q21.3) had not been described previously. Nine of the other CNAs affected known PCa tumor suppressor genes [CHD1 (5q21.1), MAPK3K7 (6q15), PTEN (10q23.31), CDKN1B (12p13.1), RB1 (13q14.2), and TP53 (17p13.1)] or oncogenes [MYC (8q24.2), TPD52 (8q21.3) and TMPRSS2-ERG fusions (21q22)]. For the other CNAs, the analyses identified or reduced the number of candidate targets. For example, LRP1B and RYBP1 appeared to be targets of the novel CNA at 2q22.1 and deletions at 3p13, respectively. It was also confirmed that deletions on 5q21.1 targeted CHD1 whereas those at 5q11.2 involved PDE4D and RAB3C.⁷

Association of CNAs with Clinicopathologic Features and Clinical Outcome.

As expected, univariate analysis showed that high tumor Gleason score (P=0.01) and stage (P=0.05) correlated with PCa-specific mortality in the JHH cohort, although PSA level and age at surgery did not (Table 2). It was also found that adjuvant treatment of a subset of patients did not reduce PCa-specific mortality. On univariate analysis, six of the twenty CNAs (TP53, MAP3K7, CHD1, PDE4D, COL1A2, and PTEN) were associated with high Gleason scores, tumor stage, or both. Importantly, CNAs of MYC (8q24.21), SERPINB5 (18q21.33), TPD52 (8q21.13), USP10 (16q24.1), PTEN (10q23.31), TP53 (17p13.1), and a novel one described here, ADAR (1q21.3), significantly correlated with PCa-specific mortality (Table 2). For five, the strength of association was similar to or greater than that observed with Gleason score or tumor stage. Gains of MYC conferred the greatest risk of dying from PCa with an OR of 4.75 (P=0.002).

Multivariate logistic regression analysis using a model that incorporated the genetic markers and clinicopathologic variables (forced-in) showed that the CNAs of PTEN and MYC conferred additional independent prognostic information. The P-value of the Hosmer-Lemeshow test for the calibration of this model was 0.703. For PTEN loss, the estimated OR for PCa-specific mortality was 7.31 [95% confidence interval (CI): 1.98-27.0; P=0.003] and for MYC gain was 7.82 (95% CI: 2.30-26.6; P=0.001) (Table 3). To explore a joint effect, distributions of CNAs at PTEN and MYC within this patient population were analyzed (Table 4). Although the presence of either conferred a borderline increase in risk, those patients whose tumors harbored both alterations had a significant and markedly increased OR for PCa-specific mortality of 53 (95% C.I.=6.92-405, P=1×10⁻⁴). Consistent with a joint effect, patients whose tumors had any combination of the two CNAs had an OR of 7.36 (95% CI: 1.57-34.5; P=0.0112). The independent prognostic information provided by the CNA data appeared to be most relevant to tumors with Gleason scores of 7 or less. Tests were conducted to determine if the AUC was significantly improved by adding genetic variables using the nonparametric method of De Long et al.¹⁰ In this group, inclusion of the genetic data in prognostic models generated by logistic regression improved the AUC by ˜12% to 0.89, which achieved borderline significance, whereas the improvement for tumors with Gleason scores of 8 or more was not significant.

Association of CNAs at PTEN/MYC with PCa-Specific Mortality in Independent Patient Cohorts and with Metastatic Disease.

To confirm the associations of PTEN and MYC alterations in tumor DNA with PCa-specific mortality, two other independent patient cohorts were studied. These included 103 patients who underwent prostatectomy at KUH (Table 1) and 216 patients treated at MSKCC. In the latter group, the clinical and comparative genomic hybridization data were retrieved from a publically accessible database.⁷ The analysis of both groups was confounded by the low number of PCa-specific deaths (four in each set) that was likely related to the inclusion of patients with favorable clinicopathologic prognostic features in the study populations. Nonetheless, in the MSKCC group, a significant association of MYC gain (P=0.0216) or CNAs at PTEN and/or MYC (P=0.0092) with PCa-specific mortality was found. The Cochran-Armitage trend test revealed a highly significant joint effect (P^{trend}=0.003). For tumors from the KUH cohort, the trend test revealed equivocal results (P=0.1071); however, when the KUH and MSKCC cohorts were analyzed together by the Cochran-Mantel-Haenszel (CMH) test, the relationship between the CNAs of PTEN/MYC and PCa-specific mortality proved to be highly significant (P=0.004). In the combined group, only a single patient out of 201 (0.5%) whose tumors lacked these markers died of PCa, in contrast to 6% for those with tumors harboring CNAs of MYC, PTEN, or both.

One inference from the above results is that PCa harboring CNAs of PTEN and MYC are more likely to have the acquired lethal phenotype and thus alterations in these genes are expected to be over-represented in lethal PCa. As a first step to address this possibility, the CNAs in tumor tissues obtained at autopsy from 14 men who died from metastatic PCa⁶ were analyzed. The results showed that tumors from all of these patients (100%) had alterations in at least one of these two genes and eight (57%) had alterations in both genes, compared to 58.4% and 9.6%, respectively, in localized PCa from the JHH prostatectomy cohort.

Among the twenty regions of significant CNAs (FIG. 1), eleven were associated with clinicopathologic risk factors or PCa-specific mortality and therefore may contribute to a more aggressive tumor phenotype. Thus, further elucidation of the molecular basis of the oncogenic effects of the newly identified and less studied CNAs may uncover new therapeutic targets. Such a strategy is exemplified by the discovery that loss of PTEN in PCa cells increases signaling in the PI3K pathway which then provided a solid rationale for clinical trials of inhibitors of this pathway.

Although seven CNAs [MYC (8q24.21), ADAR (1q21.3), SERPINB5 (18q21.33-22.1), TPD52 (8q21.13), USP10 (16q24.1), PTEN (10q23.31), and TP53 (17p13.1)] significantly correlated with early PCa-specific mortality in the JHH cohort, only those of PTEN and MYC contributed prognostic information beyond that provided by standard clinicopathologic features. The relationship of these two CNAs with clinical outcome was also evident in the MSKCC and KUH patients despite the low number of PCa-specific deaths in each group. This study is the first to demonstrate a joint effect in clinical cohorts where CNAs of both PTEN and MYC in the tumor genome imposed the most significant risk for PCa-specific mortality following prostatectomy.

An important question is whether the independent prognostic information provided by CNAs of PTEN and MYC is sufficient to impact clinical management or the stratification of patients in clinical trials. A variety of other molecular markers, including genetic alterations and gene expression profiles, have been developed and tested in hopes of improving the accuracy of prognostic models.^{11, 15, 16} Although many have shown promise, none have been sufficiently validated and/or robust to justify their clinical application. CNAs detected by SNP microarrays have also been reported to correlate with biochemical relapse following prostatectomy or radiation therapy.^{7, 17} However, biochemical relapse per se often shows little or no correlation with PCa-specific mortality.^{18, 19}

Our analyses of tumor DNAs from three additional cohorts with a total of 333 patients support an association between CNAs of PTEN and MYC with PCa-specific mortality. These genetic data could be incorporated into predictive models to help select prostatectomy patients who are more or less likely to benefit from adjuvant therapies and stratify patients for clinical studies.^{23, 24} The revised model may be particularly helpful for segregating patients with Gleason 7 tumors, a troublesome category in terms of predicting outcome. As shown here, the main impact of the CNA data on prognostic accuracy in the JHH patients was for those patients whose tumors had Gleason scores of ≦7. In addition, we noted that none of the 37 (0%) JHH patients with Gleason≦7 tumors lacking CNAs at PTEN and MYC died of PCa. Similarly only one out of 201 (0.5%) in the MSKCC and KUH cohorts, the majority of whom had Gleason≦7 tumors, died of PCa. This compares to nine of 45 (20%) and seven of 117 (6%), respectively, for those patients whose tumors contained one or both markers, that died of PCa. Thus, patients whose tumors have lower Gleason scores and lack of CNAs of PTEN and MYC are unlikely to benefit from adjuvant therapies. The prognostic significance of the CNAs detected in resected PCa may also apply to CNAs determined by FISH or SNP microarray analyses of genomic DNAs in tumor cells obtained by needle biopsy. CNA profiles in biopsy samples could help select those patients best treated with prostatectomy or radiation and those who could be managed conservatively.

### REFERENCES

1. Siegel R, Naishadham D, Jemal A. Cancer statistics, 2012. CA Cancer J Clin 2012; 62(1): 10-29.
2. Boorjian S A, Karnes R J, Viterbo R, et al. Long-term survival after radical prostatectomy versus external-beam radiotherapy for patients with high-risk prostate cancer. Cancer 2011; 117(13): 2883-2891.
3. Kibel A S, Ciezki J P, Klein E A, et al. Survival among men with clinically localized prostate cancer treated with radical prostatectomy or radiation therapy in the prostate specific antigen era. J Urol 2012; 187(4): 1259-1265.
4. Bill-Axelson A, Holmberg L, Ruutu M, et al. Radical prostatectomy versus watchful waiting in early prostate cancer. N Engl J Med 2011; 364(18): 1708-1717.
5. Carver B S, Tran J, Gopalan A, et al. Aberrant ERG expression cooperates with loss of PTEN to promote cancer progression in the prostate. Nat Genet 2009; 41(5): 619-624.
6. Liu W, Laitinen 5, Khan S, et al. Copy number analysis indicates monoclonal origin of lethal metastatic prostate cancer. Nat Med 2009; 15(5): 559-565.
7. Taylor B S, Schultz N, Hieronymus H, et al. Integrative genomic profiling of human D prostate cancer. Cancer Cell 2010; 18(1): 11-22.
8. Berger M F, Lawrence M S, Demichelis F, et al. The genomic complexity of primary human prostate cancer. Nature 2011; 470(7333): 214-220.
9. Beroukhim R, Getz G, Nghiemphu L, et al. Assessing the significance of chromosomal aberrations in cancer: methodology and application to glioma. Proc Natl Acad Sci USA 2007; 104(50): 20007-20012.
10. DeLong E R, DeLong D M, Clarke-Pearson D L. Comparing the areas under two or more correlated receiver operating characteristic curves: a nonparametric approach. Biometrics 1988; 44(3): 837-845.
11. Ding Z, Wu C J, Chu G C, et al. SMAD4-dependent barrier constrains prostate cancer growth and metastatic progression. Nature 2011; 470(7333): 269-273.
12. Lapointe J, Li C, Giacomini C P, et al. Genomic profiling reveals alternative genetic pathways of prostate tumorigenesis. Cancer Res 2007; 67(18): 8504-8510.
13. Tsuchiya N, Slezak J M, Lieber M M, Bergstralh E J, Jenkins R B. Clinical significance of alterations of chromosome 8 detected by fluorescence in situ hybridization analysis in pathologic organ-confined prostate cancer. Genes Chromosomes Cancer 2002; 34(4): 363-371.
14. Yoshimoto M, Cunha I W, Coudry R A, et al. FISH analysis of 107 prostate cancers shows that PTEN genomic deletion is associated with poor clinical outcome. Br J Cancer 2007; 97(5): 678-685.
15. Cheville J C, Karnes R J, Therneau T M, et al. Gene panel model predictive of outcome in men at high-risk of systemic progression and death from prostate cancer after radical retropubic prostatectomy. J Clin Oncol 2008; 26(24): 3930-3936.
16. Glinsky G V, Glinskii A B, Stephenson A J, Hoffman R M, Gerald W L. Gene expression profiling predicts clinical outcome of prostate cancer. J Clin Invest 2004; 113(6): 913-923.
17. Zafarana G, Ishkanian A S, Malloff C A, et al. Copy number alterations of c-MYC and PTEN are prognostic factors for relapse after prostate cancer radiotherapy. Cancer 2012.
18. Buyyounouski M K, Pickles T, Kestin L L, Allison R, Williams S G. Validating the interval to biochemical failure for the identification of potentially lethal prostate cancer, J Clin Oncol 2012; 30(15): 1857-1863.
19. Stephenson A J, Kattan M W, Eastham J A, et al. Defining biochemical recurrence of prostate cancer after radical prostatectomy: a proposal for a standardized definition. J Clin Oncol 2006; 24(24): 3973-3978.
20. Bouchard C, Marquardt J, Bras A, Medema R H, Eilers M. Myc-induced proliferation and transformation require Akt-mediated phosphorylation of FoxO proteins. EMBO J 2004; 23(14): 2830-2840.
21. Zhu J, Blenis J, Yuan J. Activation of PI3K/Akt and MAPK pathways regulates Myc-mediated transcription by phosphorylating and promoting the degradation of Mad1. Proc Natl Acad Sci USA 2008; 105(18): 6584-6589.
22. Kim J, Eltoum I E, Roh M, Wang J, Abdulkadir S A. Interactions between cells with distinct mutations in c-MYC and Pten in prostate cancer. PLoS Genet 2009; 5(7): e1000542.
23. Dorff T B, Flaig T W, Tangen C M, et al. Adjuvant androgen deprivation for high-risk prostate cancer after radical prostatectomy: SWOG 59921 study. J Clin Oncol 2011; 29(15): 2040-2045.
24. Montgomery B, Lavori P, Garzotto M, et al. Veterans Affairs Cooperative Studies Program study 553: Chemotherapy after prostatectomy, a phase III randomized study of prostatectomy versus prostatectomy with adjuvant docetaxel for patients with high-risk, localized prostate cancer. Urology 2008; 72(3): 474-480.
25. Barbieri C E, Baca S C, Lawrence M S, et al. Exome sequencing identifies recurrent SPOP, FOXA1 and MED12 mutations in prostate cancer. Nat Genet 2012; 44(6): 685-689.

### Example 2

### Genome-Wide Analysis of Prostate Tumors Reveals Genetic Markers Associated with Early Cancer-Specific Mortality Following Prostatectomy

Abstract.

Most prostate cancers are considered to be indolent (non-aggressive) and may not even require treatment. However, some of them are aggressive tumors that are characterized by uncontrolled cell proliferation resulting in cancer progression, recurrence and metastases, leading to ˜28,000 estimated deaths in 2012. Clinicopathologic parameters are strong predictors of disease recurrence, but there is no reliable marker to distinguish between those patients within each subgroup who are at high or low risk for prostate cancer specific mortality. To identify novel effectors and markers of localized but potentially life-threatening prostate cancer, DNA copy number alterations (CNAs) and nucleotide mutations were evaluated in the tumor genomes from patients who underwent prostatectomy using high resolution SNP arrays and exome sequencing, respectively. Studies were carried out to determine whether these somatic alterations can augment clinicopathologic parameters in predicting early prostate cancer specific death. Using the algorithm of Genomic Identification of Significant Targets in Cancer with a q-value of 0.01 to analyze the data from the tumor genomes of 125 patients, twenty significant regions of CNAs were identified, four of them novel, and the unique target genes of four of the altered regions were identified. By univariate analysis, seven CNAs were significantly associated with early prostate cancer specific mortality. These included gains of chromosomal regions that contain the genes MYC, ADAR, or TPD52 and losses of sequences that incorporate SERPINB5, USP10, PTEN, or TP53. On multivariate analysis, only the CNAs of PTEN and MYC contributed additional prognostic information independent of that provided by pathologic stage, Gleason score, and initial PSA level. Patients whose tumors had alterations of both genes had a markedly elevated risk of prostate cancer specific mortality (OR=53; C.I.=6.92-405, P=1×10⁻⁴). Analyses of the tumor genomes of 333 patients from three additional distinct patient cohorts confirmed the relationship between CNAs of PTEN and MYC and lethal prostate cancer. This study identifies new CNAs and genes that likely contribute to the pathogenesis of localized PCa and indicates that patients whose tumors have acquired CNAs of PTEN, MYC, or both have an increased risk of early PCa-specific mortality.

Patients and Methods.

The initial discovery cohort consisted of 125 eligible patients who underwent radical prostatectomy at Johns Hopkins Hospital (JHH) in the U.S. between 1988 and 2004 (Table 1). Many of these patients had a more aggressive form of prostate cancer (PCa). The second cohort included 103 prostatectomy patients who were treated between 2002 and 2008 with a median follow-up of about five years from the Karolinska University Hospital (KUH) in Sweden (Table 1). In contrast to the JHH cohort described above, most of the Swedish patients had a less aggressive form of PCa. A third cohort was composed of 216 patients from Memorial Sloan Kettering Cancer Center (MSKCC) with clinicopathologic and survival information publically available.

Somatic tumor and matched normal DNA from patients of the JHH and KUH cohorts was prepared and used for SNP array analysis of genome-wide CNAs. GISTIC method was used to identify significant CNAs. As the number of genes in each of the significant regions varied, the CNAs were named by the known or suspected tumor suppressor or oncogene within the altered sequences or by the first gene listed GISTIC in the region. Association of clinicopathologic variables and DNA CNAs with PCa-specific mortality was explored using logistic regression. Both univariate and multivariate analyses were incorporated in the logistic regression model. P-values were calculated using Fisher's exact test, Cochran-Armitage trend test, and Cochran-Mantel-Haenszel (CMH) test.

Genome-Wide Analysis of Tumor DNA Reveals the 20 Most Significant CNAs in Clinically Localized PCa.

Using the SNP arrays and GISTIC algorithm with a q-value of 0.01 to analyze 125 primary tumors from the JHH discovery cohort, the 20 most significant CNAs were identified, along with the most commonly gained or deleted gene(s) within each region (FIG. 1). Fifteen of these CNAs represented chromosomal deletions and five were gains or amplification.

Four of the CNAs, consisting of deletions that involved DISC1 (1q42.2), LRP1B (2q22.1) and HRT3A (11q23) or gains of ADAR1 (1q21.3) had not been described previously. Nine of the other CNAs affected known PCa tumor suppressor genes [CHD1 (5q21.1), MAPK3K7 (6q15), PTEN (10q23.31), CDKN1B (12p13.1), RB1 (13q14.2), and TP53 (17p13.1)] or oncogenes [MYC (8q24.2), TPD52 (8q21.3) and TMPRSS2-ERG fusions (21q22)]. For the other CNAs, the analyses identified or reduced the number of candidate targets. For example, LRP1B and RYBP1 appeared to be targets of the novel CNA at 2q22.1 and deletions at 3p13, respectively. It was also confirmed that deletions on 5q21.1 targeted CHD1 whereas those at 5q11.2 involved PDE4D and RAB3C.

Association of CNAs with Clinicopathologic Features and Clinical Outcome.

As expected, univariate analysis showed that high tumor Gleason score (P=0.01) and stage (P=0.05) correlated with PCa-specific mortality in the JHH cohort, although PSA level and age at surgery did not (Table 2). It was also found that adjuvant treatment of a subset of patients did not reduce PCa-specific mortality. On univariate analysis, six of the twenty CNAs (TP53, MAP3K7, CHD1, PDE4D, COL1A2, and PTEN) were associated with high Gleason scores, tumor stage, or both. Importantly, CNAs of MYC (8q24.21), SERPINB5 (18q21.33), TPD52 (8q21.13), USP10 (16q24.1), PTEN (10q23.31), TP53 (17p13.1), and a novel one described here, ADAR (1q21.3), significantly correlated with PCa-specific mortality (Table 2). Gains of MYC) conferred the greatest risk of dying from PCa with an OR of 4.75 (P=0.002).

Multivariate logistic regression analysis using a model that incorporated the genetic markers and clinicopathologic variables (forced-in) showed that the CNAs of PTEN and MYC conferred additional independent prognostic information. For PTEN loss, the estimated OR for PCa-specific mortality was 7.31 [95% confidence interval (CI): 1.98-27.0; P=0.003] and for MYC gain was 7.82 (95% CI: 2.30-26.6; P=0.001) (Table 3). To explore a joint effect, the distributions of CNAs at PTEN and MYC within this patient population were analyzed (Table 4). Although the presence of either conferred a borderline increase in risk, those patients whose tumors harbored both alterations had a significant and markedly increased OR for PCa-specific mortality of 53 (95% C.I.=6.92-405, P=1×10⁻⁴). Consistent with a joint effect, patients whose tumors had any combination of the two CNAs had an OR of 7.36 (95% CI: 1.57-34.5; P=0.0112).

Independent Prognostic Information Provided by the CNA Data Appears to be Most Relevant to Tumors with Gleason Scores of 7 or Less.

Studies were conducted to determine whether the receiver operating characteristic curve (AUC) was significantly improved by adding genetic variables using the nonparametric method of De Long et al. In this group, inclusion of the genetic data in prognostic models generated by logistic regression improved the AUC by ˜27% to 0.88 which achieved statistical significance, whereas the improvement for tumors with Gleason scores of 8 or more was not significant (Table 5).

The model may be particularly helpful for segregating patients with Gleason 7 tumors, a troublesome category in terms of predicting outcome. As shown here, the main impact of the CNA data on prognostic accuracy in the JHH patients was for those patients whose tumors had Gleason scores of ≦7. In addition, it was noted that none of the 37 (0%) JHH patients with Gleason≦7 tumors lacking CNAs at PTEN and MYC died of PCa.

Association of CNAs at PTEN/MYC with PCa-Specific Mortality in Independent Patient Cohorts and with Metastatic Disease.

To confirm the associations of PTEN and MYC alterations in tumor DNA with PCa-specific mortality, two other independent patient cohorts were studied. These included 103 patients who underwent prostatectomy at KUH (Table 1) and 216 patients treated at MSKCC. In the later group, the clinical and CGH data were retrieved from a publically accessible database. The analysis of both groups was confounded by the low number of PCa-specific deaths (four in each set) that was likely related to the inclusion of patients with favorable clinicopathologic prognostic features in the study populations (Table 6). Nonetheless, in the MSKCC group, a significant association of MYC gain (P=0.0216) or CNAs at PTEN and/or MYC (P=0.0092) with PCa-specific mortality was found. The Cochran-Armitage trend test revealed a highly significant joint effect (P^{trend}=0.003). For tumors from the KUH cohort, the trend test revealed equivocal results (P=0.1071); however, when the KUH and MSKCC cohorts were analyzed together by the Cochran-Mantel-Haenszel (CMH) test, the relationship between the CNAs of PTEN/MYC and PCa-specific mortality proved to be highly significant (P=0.004). In the combined group, only a single patient out of 201 (0.5%) whose tumors lacked these markers died of PCa, in contrast to 6% for those with tumors harboring CNAs of MYC, PTEN, or both.

One inference from the above results is that PCa harboring CNAs of PTEN and MYC are more likely to have the acquired lethal phenotype and thus alterations in these genes are expected to be over-represented in lethal PCa. As a first step to address this possibility, the CNAs in tumor tissues obtained at autopsy from 14 men who died from metastatic PCa at JHH were analyzed. The results showed that tumors from all of these patients (100%) had alterations in at least one of these two genes and eight (57%) had alterations in both genes, compared to 58.4% and 9.6%, respectively, in localized PCa from the JHH prostatectomy cohort.

All publications and patent applications, nucleotide sequences and/or amino acid sequences identified by GenBank® Database Accession numbers are herein incorporated by reference to the same extent as if each individual publication or patent application or sequences was specifically and individually indicated to be incorporated by reference.

Although the foregoing invention has been described in some detail by way of illustration and example for purposes of clarity of understanding, it will be apparent that certain changes and modifications may be practiced within the scope of the list of the foregoing embodiments and the appended claims.

TABLE 1
Clinical information of study subjects in the cohorts from Johns Hopkins
Hospital in the US and Karolinska University Hospital in Sweden

| | JHH | | KUH | |
|---|---|---|---|---|
| | Not died of | Died of | Not died of | Died of |
| | prostate | prostate | prostate | prostate |
| Characteristics | cancer (103) | cancer (22) | cancer (99) | cancer (4) |
| Age | | | | |
| Mean(std) | 59.74(7.09) | 58.1(7.34) | 63.7(4.67) | 67.8(5.62) |
| Min-Max | 36-74 | 39-71 | 54-75 | 63-74 |
| Preoperational | | | | |
| PSA (ng/ml) | | | | |
| <4 | 7(6.8%) | 3(13.6%) | 6(6.0%) | 1(25%) |
| 4-10 | 44(42.7%) | 8(36.4%) | 56(56.6%) | 3(75%) |
| >10 | 46(44.7%) | 9(40.9%) | 29(29.3%) | 0(0%) |
| NA | 6(5.8%) | 2(9.1%) | 8(8.1%) | 0(0%) |
| Gleason grade | | | | |
| 5 | 1(1.0%) | 1(4.5%) | 6(6.1%) | 0(0%) |
| 6 | 20(19.4%) | 0(0.0%) | 42(42.4%) | 2(50%) |
| 7 | 52(50.5%) | 8(36.4%) | 37(37.4%) | 1(25%) |
| 8 | 15(14.6%) | 5(22.7%) | 8(8.1%) | 1(25%) |
| 9 | 14(13.6%) | 8(36.4%) | 2(2.0%) | 0(0%) |
| 10 | 1(1.0%) | 0(0.0%) | 0(0%) | 0(0%) |
| NA | | | 4(4.1%) | 0(0%) |
| Clinical stage | | | | |
| T1 | 0(0%) | 0(0%) | 42(42.4%) | 0(0%) |
| T2 | 11(10.7%) | 1(4.5%) | 39(39.4%) | 3(75%) |
| T3A | 60(58.3%) | 9(40.9%) | 9(9.1%) | 1(25%) |
| T3B | 30(29.1%) | 11(50.0%) | 0(0%) | 0(0%) |
| NA | 2(1.9%) | 1(4.5%) | 9(9.1%) | 0(0%) |

TABLE 2
Association of clinicopathological variables and DNA copy number changes with PCa-specific death

| | | Number of subjects (%) | | Logistic regression | |
|---|---|---|---|---|---|
| | | PCa-specific death | Remaining patients | | P |
| Variables | Values^{†} | (N = 22) | (N = 103) | OR (95% CI) | value |
| Clinicopathologic Variables | | | | | |
| Age at surgery | | | | 0.97(0.91-1.03) | 0.339 |
| Gleason Score | ≧8 | 13(59) | 30(29) | 3.51(1.36-9.09) | 0.010 |
| Tumor-Stage | ≧T3b | 11(50) | 30(29) | 2.60(1.00-6.78) | 0.050 |
| Preoperative PSA | ≧10 ng/mL | 9(41) | 46(45) | 0.91(0.35-2.39) | 0.843 |
| Somatic DNA copy number | | | | | |
| changes at candidate genes | | | | | |
| MYC (8q24.21) | g, a | 13(59) | 24(23) | 4.75(1.81-12.48) | 0.002 |
| ADAR (1q21.3) | a, g | 6(27) | 6(6) | 6.06(1.74-21.14) | 0.005 |
| SERPINB5 (18q21.33-22.1) | d | 10(45) | 19(18) | 3.68(1.39-9.78) | 0.009 |
| TPD52 (8q21.13) | g, a | 12(55) | 27(26) | 3.38(1.31-8.71) | 0.012 |
| USP10 (16q24.1) | d, dd | 15(68) | 39(38) | 3.52(1.32-9.38) | 0.012 |
| PTEN (10q23.31) | d, dd | 13(59) | 35(34) | 2.81(1.09-7.20) | 0.032 |
| TP53 (17p13.1) | d | 10(45) | 30(29) | 2.60(1.00-6.74) | 0.049 |
| PDE4D (5q11.2) | d | 7(32) | 19(18) | 2.06(0.74-5.76) | 0.167 |
| TMPRSS2-ERG (21q22.2-22.3) | d | 9(41) | 25(24) | 1.95(0.75-5.07) | 0.172 |
| COL1A2 (7q21.3) | a, g | 7(32) | 21(20) | 1.82(0.66-5.04) | 0.248 |
| HTR3A (11q23.2) | d, dd | 5(23) | 14(14) | 1.87(0.59-5.88) | 0.284 |
| CHD1 (5q21.1) | d, dd | 8(36) | 26(25) | 1.69(0.64-4.49) | 0.291 |
| RYBP (3p13) | d | 3(14) | 20(19) | 0.66(0.18-2.43) | 0.528 |
| DISC1 (1q42.2) | d | 3(14) | 19(18) | 0.70(0.19-2.60) | 0.593 |
| LRP1B (2q22.1) | d | 5(23) | 19(18) | 1.30(0.43-3.96) | 0.644 |
| CDKN1B (12p13.1) | d, dd | 7(32) | 30(29) | 1.14(0.42-3.06) | 0.802 |
| MAP3K7 (6q15) | d, dd | 10(45) | 44(43) | 1.12(0.44-2.82) | 0.814 |
| RB1 (13q14.2) | d, dd | 10(45) | 44(43) | 1.12(0.44-2.82) | 0.814 |
| ATP1B3 (3q23) | a, g | 3(14) | 16(16) | 0.86(0.23-3.24) | 0.822 |
| BNIP3L (8p21.2) | d, dd | 13(59) | 62(60) | 0.96(0.37-2.44) | 0.923 |

^{†}Clinicopathological variables are dichotomized at the cut-off point of presented values. ‘d’ and ‘dd’ denote hemizygous and homozygous deletion, respectively. ‘g’ and ‘a’ denote one and > one additional copy gain of DNA, respectively.

TABLE 3
Multivariate analysis using clinicopathological
variables and copy number changes†

| Variables | Values‡ | OR (95% CI) | P value |
|---|---|---|---|
| Clinicopathologic Variables | | | |
| Age at surgery | | 0.95(0.88-1.03) | 0.188 |
| Gleason score | ≧8 | 3.42(1.10-10.7) | 0.034 |
| Preoperative PSA | ≧10 ng/mL | 1.25(0.39-4.02) | 0.714 |
| Somatic DNA copy number | | | |
| changes at candidate genes | | | |
| PTEN (10q23.31) | d, dd | 7.31(1.98-27.0) | 0.003 |
| MYC (8q24.21) | g, a | 7.82(2.30-26.6) | 0.001 |

†Using the genes with P value < 0.05 in Table 1, step-wise selection was used for selecting candidate genes. Clinicopathological variables were forced into the model during model selection.
‡Clinicopathological variables are dichotomized at the cut-off point of presented values. d’ and ‘dd’ denote hemizygous and homozygous deletion, respectively. ‘g’ and ‘a’ denote one and > one additional copy gain of DNA, respectively.

TABLE 4
Joint effect of DNA copy number changes at PTEN and MYC on PCa-specific death†

| | | | Number of subjects (%) | | | |
|---|---|---|---|---|---|---|
| | | | PCa-specific | Remaining | | |
| Deletion | | Gain at | death | patients | | P |
| at PTEN | | MYC | (N = 22) | (N = 103) | OR (95% CI) | value |
| No | | No | 3(14) | 49(48) | 1.00 | |
| | | | | | (reference) | |
| Yes | and | No | 6(27) | 30(29) | 4.85 | 0.0759 |
| | | | | | (0.85-27.7) | |
| No | and | Yes | 6(27) | 19(18) | 5.03 | 0.0734 |
| | | | | | (0.86-29.4) | |
| Yes | and | Yes | 7(32) | 5(5) | 53.0 | 0.0001 |
| | | | | | (6.92-405.6) | |
| Yes | and/or* | Yes | 19(86) | 54(52) | 7.36 | 0.0112 |
| | | | | | (1.57-34.5) | |

†Logistic regression was performed with adjustment for clinicopathological variables in Table 2.
*and/or denotes any alteration at PTEN and MYC.

TABLE 5
Analysis of the area under the receiver operating characteristic
curve (AUC) using clinicopathoiogical and CNAs

| | Without T-stage* | | |
|---|---|---|---|
| Variable | AUC | Difference | P-value |
| age, PSA, Gleason | 0.659 | reference | 0.0407 |
| plus PTEN + MYC | 0.820 | 0.161 | |
| Gleason < 8 | | | |
| age, PSA | 0.606 | reference | 0.0038 |
| plus PTEN + MYC | 0.879 | 0.273 | |
| Gleason ≧ 8 | | | |
| age, PSA | 0.750 | reference | 0.1485 |
| plus PTEN + MYC | 0.864 | 0.114 | |

*T-stage and Gleason score contribute similar effect on the calculation of AUC for predicting PCa specific death in JHH cohort.

TABLE 6
Validation of the association between CNAs at PTEN/MYC and prostate cancer specific
death in two additional cohorts

| | | | MSKCC | | | KUH | | | |
|---|---|---|---|---|---|---|---|---|---|
| | | | PCa | | | PCa | | | |
| | | | specific | Remaining | P value | specific | Remaining | P value | P value |
| Deletion | | Gain at | death | patients | (Fisher | death | patients | (Fisher | (CMH |
| at PTEN | | MYC | (N = 4) | (N = 212) | test) | (N = 4) | (N = 99) | test) | test) |
| No | | No | 0 | 148 | | 1 | 53 | | |
| Yes | and | No | 1 | 25 | 0.1494 | 1 | 29 | 1.0000 | 0.1359 |
| No | and | Yes | 2 | 24 | 0.0216 | 1 | 9 | 0.2902 | 0.0008 |
| Yes | and | Yes | 1 | 15 | 0.0976 | 1 | 8 | 0.2673 | 0.0048 |
| Yes | *and/or | Yes | 4 | 64 | 0.0092 | 3 | 46 | 0.3445 | 0.0044 |
| Trend | | | | | 0.0030 | | | 0.1071 | |
| test | | | | | | | | | |

TABLE 7
Significant deletion genes identified by GISTIC with a q-value of 0.25 and a join segment size of 60

| | | Residual | Wide peak | | | | | | | |
|---|---|---|---|---|---|---|---|---|---|---|
| Cytoband | q value | q value | boundaries* | Genes in wide peak | | | | | | |
| 1p21.3 | 0.22145 | 0.22145 | chr1:94623418- | CNN3 | F3 | ABCD3 | RWDD3 | SLC44A3 | TMEM56 | ALG14 |
| | | | 95506264 | | | | | | | |
| 1q42.2 | 0.0023055 | 0.0023055 | chr1:229894700- | DISC1 | SIPA1L2 | | | | | |
| | | | 230947362 | | | | | | | |
| 2p21 | 0.1628 | 0.22145 | chr2:42798784- | ZFP36L2 | HAAO | THADA | PLEKHH2 | OXER1 | | |
| | | | 43727008 | | | | | | | |
| 2q22.1 | 6.84E−08 | 6.84E−08 | chr2:139707778- | LRP1B | | | | | | |
| | | | 140858852 | | | | | | | |
| 3p13 | 3.20E−07 | 3.20E−07 | chr3:72106391- | RYBP | | | | | | |
| | | | 72637217 | | | | | | | |
| 4p15.1 | 0.068974 | 0.068974 | chr4:32895232- | CENTD1 | | | | | | |
| | | | 34441989 | | | | | | | |
| 4q34.3 | 0.096805 | 0.19065 | chr4:180521165- | LOC285501 | | | | | | |
| | | | 180694787 | | | | | | | |
| 5q11.2 | 1.02E−08 | 1.85E−05 | chr5:58150917- | PDE4D | RAB3C | | | | | |
| | | | 58594798 | | | | | | | |
| 5q21.1 | 2.46E−19 | 2.46E−19 | chr5:98160357- | CHD1 | | | | | | |
| | | | 98265501 | | | | | | | |
| 6q15 | 1.84E−27 | 1.84E−27 | chr6:90489384- | MAP3K7 | CASP8AP2 | MDN1 | BACH2 | GJA10 | | |
| | | | 91304420 | | | | | | | |
| 8p21.2 | 5.16E−48 | 5.16E−48 | chr8:25894784- | BNIP3L | PPP2R2A | EBF2 | | | | |
| | | | 26348955 | | | | | | | |
| 8q24.23 | 0.025882 | 0.03881 | chr8:137747079- | KHDRBS3 | | | | | | |
| | | | 137931364 | | | | | | | |
| 10q23.31 | 5.16E−48 | 5.16E−48 | chr10:89843423- | PTEN | | | | | | |
| | | | 89888562 | | | | | | | |
| 11q23.2 | 0.000253 | 0.000253 | chr11:113321588- | HTR3A | NNMT | ZBTB16 | HTR3B | RBM7 | REXO2 | C11orf71 |
| | | | 113946501 | FAM55A | | | | | | |
| 12p13.1 | 8.10E−14 | 8.10E−14 | chr12:12740509- | hsa-mir-613 | CDKN1B | APOLD1 | | | | |
| | | | 12846005 | | | | | | | |
| 12q24.33 | 0.1373 | 0.1373 | chr12:130844146- | MMP17 | SFRS8 | ULK1 | EP400 | NOC4L | PUS1 | DDX51 |
| | | | 131213322 | | | | | | | |
| 13q14.2 | 2.97E−27 | 2.97E−27 | chr13:47881690- | RCBTB2 | RB1 | P2RY5 | CYSLTR2 | | | |
| | | | 48401865 | | | | | | | |
| 15q21.3 | 0.082642 | 0.082642 | chr15:54755414- | TCF12 | ZNF280D | | | | | |
| | | | 55297252 | | | | | | | |
| 16q24.1 | 3.41E−21 | 3.41E−21 | chr16:82877051- | USP10 | ATP2C2 | COTL1 | KIAA1609 | WFDC1 | C16orf44 | CRISPLD2 |
| | | | 83540927 | | | | | | | |
| 17p13.1 | 2.53E−13 | 2.53E−13 | chr17:7501561- | ATP1B2 | CHD3 | EFNB3 | TP53 | KCNAB3 | JMJD3 | WDR79 |
| | | | 7781403 | TRAPPC1 | LSMD1 | TMEM88 | CNTROB | CYB5D1 | DNAH2 | |
| 17q21.31 | 0.013865 | 0.021114 | chr17:39458237- | GRN | ITGA2B | SLC4A1 | UBTF | HDAC5 | RLTNDC3A | GPATCH8 |
| | | | 39902399 | SLC25A39 | ATXN7L3 | C17orf53 | TMUB2 | G6PC3 | ASB16 | LSM12 |
| | | | | C17orf65 | | | | | | |
| 18q22.1 | 1.92E−06 | 1.92E−06 | chr18:58288577- | BCL2 | KDSR | SERPINB2 | SERPINB5 | SERPINB8 | SERPINB10 | SERPINB13 |
| | | | 60834535 | SERPINB3 | SERPINB4 | SERPINB7 | VPS4B | PHLPP | ZCCHC2 | SERPINB12 |
| | | | | SERPINB11 | | | | | | |
| 21q22.3 | 4.97E−12 | 4.97E−12 | chr21:41650304- | MX1 | MX2 | TMPRSS2 | FAM3B | | | |
| | | | 41762041 | | | | | | | |

*The physical location of wide peak boundaries is based on hg18.

TABLE 8
Significant deletion genes identified by GISTIC with a q-value of 0.01 and a join segment size of 80

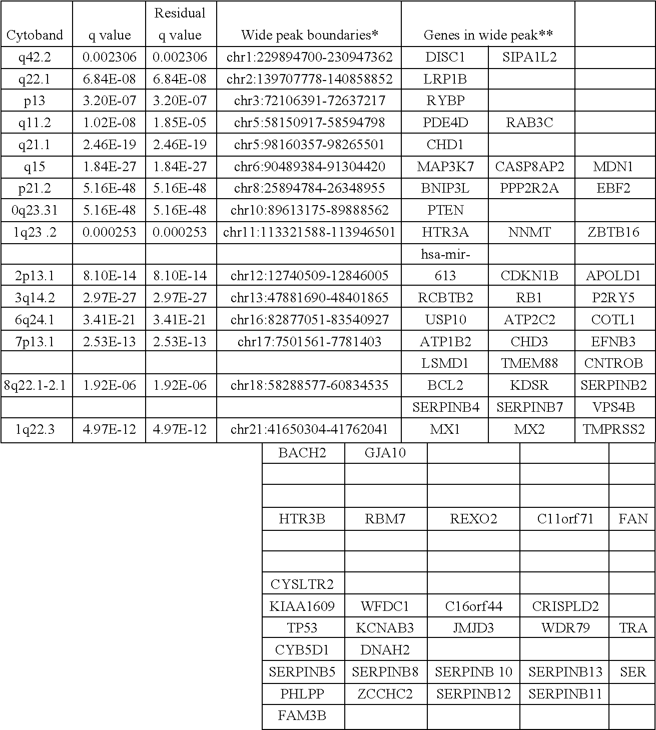

*The physical location of wide peak boundaries is based on hg18.
**Highlighted genes were chosen to represent each of these significant regions in subsequent analysis of the associations between CNAs and clinical outcomes.

TABLE 9
Significant amplification genes identified by GISTIC with a q-value of 0.25 and a join segment size of 60

| Cyto- | | Residual q | Wide peak | | | | | | | | |
|---|---|---|---|---|---|---|---|---|---|---|---|
| band | q value | value | boundaries | Genes in wide peak | | | | | | | |
| 1q21.3 | 0.0003427 | 0.00034273 | chr1: | ADAR | CHRNB2 | CKS1B | KCNN3 | SHC1 | PMVK | ZBTB7B | UBE2Q1 |
| | | | 152725557- | LENEP | PBXIP1 | FLAD1 | PYGO2 | TDRD10 | SHE | DCST2 | DCST1 |
| | | | 153275233 | | | | | | | | |
| 3p243 | 0.051033 | 0.14879 | chr3: | RAB5A | RARB | RPL15 | TGEBR2 | THRB | TOP2B | UBE2E1 | UBE2E2 |
| | | | 19393738- | EOMES | PCAF | SLC4A7 | NR1D2 | RBMS3 | NKIRAS1 | OXSM | NGLY1 |
| | | | 30800328 | AZI2 | ZNF385D | LRRC3B | KCNH8 | SGOL1 | EFHB | ZCWPW2 | C3orf68 |
| | | | | NEK10 | GADL1 | | | | | | |
| 3q23 | 9.25E−08 | 9.25E−08 | chr3: | ATP1B3 | ATR | PLS1 | RASA2 | RBP1 | RBP2 | TFDP2 | TRPC1 |
| | | | 140202645- | COPB2 | CHST2 | RNF7 | SR140 | PCOLCE2 | XRN1 | SLC25A36 | MRPS22 |
| | | | 146503982 | CLSTN2 | SPSB4 | ACPL2 | GRK7 | C3orf58 | ZBTB38 | GK5 | SLC9A9 |
| | | | | TRIM42 | PAQR9 | NMNAT3 | LOC389151 | | | | |
| 7p22.2 | 3.50E−07 | 0.14879 | chr7: | KIAA0415 | RADIL | PAPOLB | RBAK | SDK1 | FOXK1 | MMD2 | |
| | | | 4239372- | | | | | | | | |
| | | | 5092747 | | | | | | | | |
| 7q121.3 | 1.52E−10 | 1.52E−10 | chr7: | COL1A2 | GNG11 | GNGT1 | PON1 | PON2 | PON3 | TFPI2 | SGCE |
| | | | 93282152- | BET1 | PEG10 | PPP1R9A | CASD1 | | | | |
| | | | 94898177 | | | | | | | | |
| 7q36.2 | 1.73E−05 | 0.11668 | chr7: | HTR5A | PAXIP1 | | | | | | |
| | | | 154362054- | | | | | | | | |
| | | | 154559361 | | | | | | | | |
| 8q11.1 | 5.33E−11 | 0.11668 | chr8: | A26A1 | | | | | | | |
| | | | 43533313- | | | | | | | | |
| | | | 47504127 | | | | | | | | |
| 8q21.13 | 3.69E−37 | 3.69E−37 | chr8: | TPD52 | ZBTB10 | LOC389672 | ZNF704 | | | | |
| | | | 81128386- | | | | | | | | |
| | | | 08186795 | | | | | | | | |
| 8q24.21 | 2.20E−35 | 5.04E−09 | chr8: | MYC | | | | | | | |
| | | | 128095593- | | | | | | | | |
| | | | 129190507 | | | | | | | | |

* The physical location of wide peak boundaries is based on hg18.

TABLE 10
Significant amplification genes identified by GISTIC with a q-value of 0.01 and a join segment size of 80.

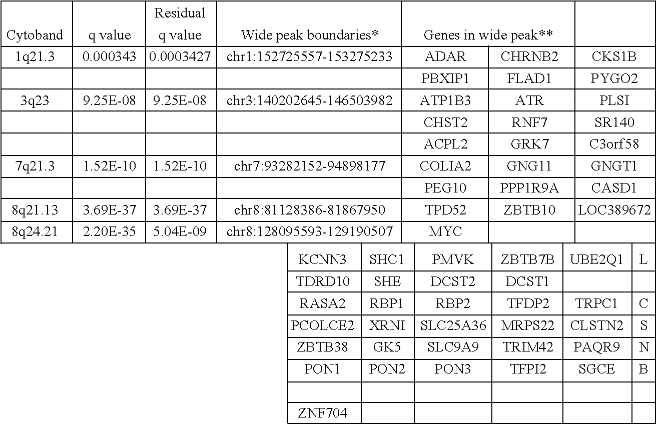

*The physical location of wide peak boundaries is based on hg18
**Highlighted genes were chosen to represent each of these significant regions in subsequent analyses of the association between CNAs and clinical outcomes.

TABLE 11
Reference genomic regions for detecting CNAs

| Chromosome | Cytoband | Start | End | Length |
|---|---|---|---|---|
| 1 | 1p32.2 | 58,245,873 | 58,266,757 | 20,884 |
| 1 | 1p13.2 | 111,622,167 | 111,622,340 | 173 |
| 1 | 1p13.2 | 112,523,438 | 112,541,094 | 17,656 |
| 1 | 1q21.3 | 150,872,722 | 150,907,586 | 34,864 |
| 1 | 1q21.3 | 151,062,352 | 151,064,221 | 1,869 |
| 1 | 1q23.1 | 156,754,150 | 156,754,299 | 149 |
| 1 | 1q24.3 | 170,691,993 | 170,716,718 | 24,725 |
| 1 | 1q25.2 | 176,892,122 | 176,895,634 | 3,512 |
| 1 | 1q25.2 | 177,344,638 | 177,344,755 | 117 |
| 1 | 1q43 | 238,514,073 | 238,519,857 | 5,784 |
| 2 | 2p25.1 | 7,172,590 | 7,173,027 | 437 |
| 2 | 2p21 | 41,753,730 | 41,755,180 | 1,450 |
| 2 | 2p16.3 | 51,147,434 | 51,182,653 | 35,219 |
| 2 | 2p12 | 77,856,791 | 77,858,396 | 1,605 |
| 2 | 2q37.3 | 237,507,719 | 237,508,336 | 617 |
| 3 | 3p24.3 | 16,192,720 | 16,204,842 | 12,122 |
| 3 | 3p22.1 | 41,254,749 | 41,255,086 | 337 |
| 4 | 4p12 | 47,846,432 | 47,853,244 | 6,812 |
| 4 | 4q13.3 | 70,451,131 | 70,455,669 | 4,538 |
| 4 | 4q28.3 | 138,385,612 | 138,390,341 | 4,729 |
| 4 | 4q31.1 | 140,245,613 | 140,245,718 | 105 |
| 4 | 4q32.2 | 162,138,926 | 162,140,862 | 1,936 |
| 4 | 4q35.1 | 186,934,488 | 186,942,771 | 8,283 |
| 5 | 5p13.2 | 38,366,085 | 38,377,455 | 11,370 |
| 6 | 6p25.1 | 5,069,030 | 5,078,431 | 9,401 |
| 6 | 6p22.3 | 18,311,073 | 18,332,522 | 21,449 |
| 6 | 6p22.3 | 18,368,602 | 18,369,390 | 788 |
| 6 | 6p21.31 | 36,423,220 | 36,430,448 | 7,228 |
| 6 | 6q24.3 | 148,817,988 | 148,834,310 | 16,322 |
| 6 | 6q27 | 169,230,709 | 169,244,979 | 14,270 |
| 9 | 9p24.3 | 1,342,961 | 1,372,861 | 29,900 |
| 9 | 9p24.3 | 1,449,445 | 1,450,032 | 587 |
| 9 | 9p24.1 | 7,152,351 | 7,176,531 | 24,180 |
| 9 | 9p23 | 9,039,072 | 9,044,621 | 5,549 |
| 9 | 9p23 | 9,786,116 | 9,786,608 | 492 |
| 9 | 9q21.11 | 71,272,162 | 71,275,819 | 3,657 |
| 9 | 9q21.13 | 78,765,858 | 78,779,172 | 13,314 |
| 9 | 9q22.32 | 97,289,096 | 97,289,777 | 681 |
| 9 | 9q31.1 | 106,423,726 | 106,424,116 | 390 |
| 9 | 9q33.1 | 119,552,402 | 119,588,487 | 36,085 |
| 9 | 9q33.2 | 125,731,336 | 125,735,591 | 4,255 |
| 10 | 10p14 | 11,141,688 | 11,148,383 | 6,695 |
| 11 | 11p15.4 | 4,671,187 | 4,673,365 | 2,178 |
| 11 | 11p15.4 | 4,881,743 | 4,884,114 | 2,371 |
| 11 | 11p15.4 | 5,787,516 | 5,792,979 | 5,463 |
| 11 | 11p14.2 | 26,512,971 | 26,518,069 | 5,098 |
| 11 | 11p12 | 37,106,373 | 37,112,991 | 6,618 |
| 11 | 11q14.2 | 85,583,115 | 85,588,915 | 5,800 |
| 11 | 11q22.1 | 98,435,433 | 98,439,119 | 3,686 |
| 11 | 11q22.1 | 100,491,375 | 100,492,287 | 912 |
| 11 | 11q24.1 | 122,326,244 | 122,339,211 | 12,967 |
| 11 | 11q25 | 130,932,063 | 130,940,039 | 7,976 |
| 12 | 12q23.3 | 107,332,064 | 107,346,952 | 14,888 |
| 13 | 13q12.2 | 27,568,039 | 27,568,433 | 394 |
| 13 | 13q12.3 | 30,559,825 | 30,571,689 | 11,864 |
| 14 | 14q23.2 | 63,782,783 | 63,785,718 | 2,935 |
| 15 | 15q26.2 | 92,696,434 | 92,704,838 | 8,404 |
| 15 | 15q26.3 | 99,735,646 | 99,755,001 | 19,355 |
| 15 | 15q26.3 | 99,764,132 | 99,782,862 | 18,730 |
| 16 | 16p13.2 | 7,151,576 | 7,168,307 | 16,731 |
| 16 | 16p13.12 | 12,613,855 | 12,614,027 | 172 |
| 16 | 16p13.12 | 12,632,490 | 12,633,389 | 899 |
| 16 | 16p12.1 | 25,632,410 | 25,646,778 | 14,368 |
| 18 | 18p11.22 | 8,539,105 | 8,556,679 | 17,574 |
| 20 | 20q12 | 37,658,317 | 37,675,617 | 17,300 |
| 21 | 21q21.1 | 19,568,304 | 19,568,807 | 503 |
| 21 | 21q21.2 | 23,334,887 | 23,336,363 | 1,476 |
| 21 | 21q22.2 | 38,709,972 | 38,717,241 | 7,269 |
| 22 | 22q12.1 | 26,341,034 | 26,350,228 | 9,194 |

1. A method of identifying a human subject as having an increased risk of having or developing aggressive prostate cancer or as having an increased likelihood of having or developing prostate cancer, comprising detecting in a nucleic acid sample from the subject 1) a deletion at 1q42.2 from chr1:229894700-230947362 bp, 2) a deletion at 2q22.1 from chr2:139707778-140858852 bp, 3) a deletion at 11q23 from chr11:113321588-113946501 bp, 4) an amplification at 1q21.3 from chr1:152725557-153275233 bp, or 5) any combination of 1-4 above, wherein the detection of same identifies the subject as having an increased risk of having or developing aggressive prostate cancer or as having an increased likelihood of having or developing prostate cancer.

2. (canceled)

3. A method of identifying a human subject as having an increased risk of having or developing aggressive prostate cancer or as having an increased likelihood of prostate cancer-specific death, comprising detecting in a nucleic acid sample from the subject 1) a copy number alteration at 8q24.21 from chr8:128095593-129190507 bp, 2) a copy number alteration at 1q21.3 from chr1:152725557-153275233 bp, 3) a copy number alteration at 18q21.33-22.1 from chr18:58288577-60834535 bp, 4) a copy number alteration at 8q21.13 from chr8:81128386-81867950 bp, 5) a copy number alteration at16q24.1 from chr16:82877051-83540927 bp, 6) a copy number alteration at10q23.31 from chr10:89613175-89888562 bp, 7) a copy number alteration at 17p13.1 from chr17:7501561-7781403 bp, and 8) any combination thereof, wherein the detection of same identifies the subject as having an increased risk of having or developing aggressive prostate cancer or as having an increased likelihood of prostate cancer-specific death.

4. (canceled)

5. A method of identifying a human subject as having an increased risk of having or developing aggressive prostate cancer or as having an increased likelihood of prostate cancer-specific death, comprising detecting in a nucleic acid sample from the subject 1) a copy number alteration in the MYC gene, 2) a copy number alteration in the ADAR gene, 3) a copy number alteration in the SERPIN5 gene, 4) a copy number alteration in the TPD52 gene, 5) a copy number alteration in the USP10 gene, 6) a copy number alteration in the PTEN gene, 7) a copy number alteration the TP53 gene, and 8) any combination thereof, wherein the detection of same identifies the subject as having an increased risk of having or developing aggressive prostate cancer or as having an increased likelihood of prostate-cancer specific death.

6. (canceled)

7. A method of identifying a human subject as having an increased risk of having or developing aggressive prostate cancer or as having an increased likelihood of prostate cancer-specific death, comprising detecting in a nucleic acid sample from the subject a deletion in the PTEN gene and amplification of the MYC gene.

8. (canceled)

9. The method of claim 1, wherein the detecting step is carried out by comparative genomic hybridization such as metaphase and BAC/oligo/cDNA/single nucleotide polymorphic (SNP) hybridization with various resolutions.

10. The method of claim 1, wherein the detecting step is carried out using fluorescent in situ hybridization.

11. The method of claim 1, wherein the detecting step is carried out using an amplification reaction.

12. The method of claim 11, wherein the amplification reaction is quantitative polymerase chain reaction (PCR).

13. The method of claim 11, wherein the amplification reaction is a multiplex ligation-dependent probe amplification.

14. A computer-assisted method of identifying a proposed treatment and/or management for aggressive prostate cancer as an effective and/or appropriate treatment and/or management for a subject carrying a genetic marker correlated with aggressive prostate cancer, comprising the steps of:
(a) storing a database of biological data for a plurality of subjects, the biological data that is being stored including for each of said plurality of subjects:
(i) a treatment type,
(ii) at least one genetic marker associated with aggressive prostate cancer, and
(iii) at least one disease progression measure for prostate cancer from which treatment efficacy can be determined; and then
(b) querying the database to determine the dependence on said genetic marker of the effectiveness of a treatment type in treating prostate cancer, thereby identifying a proposed treatment as an effective and/or appropriate treatment for a subject carrying a genetic marker correlated with prostate cancer.

15. A kit comprising reagents to detect the copy number alteration (CAN) according to the method of claim 3 in a nucleic acid sample.

16. A kit comprising reagents to detect the copy number alteration (CNA) according to the method of claim 5 in a nucleic acid sample.

17. A kit comprising reagents to detect the copy number alteration (CNA) according to the method of claim 7 in a nucleic acid sample.
